(19) United States
(12) Patent Application Publication
(10) Pub. No.: US 2025/0169897 A1
(43) Pub. Date: May 29, 2025

(54) MEDICAL APPARATUS WITH CONTINUOUS SUPPORT STRUCTURE AND METHOD OF USE THEREOF

(71) Applicant: CANON U.S.A., INC., MELVILLE, NY (US)

(72) Inventors: Anne Yujin Edge, Somerville, MA (US); Inderpal Singh, Norwood, MA (US); Stefanos Podogiros, Kingston, NH (US)

(21) Appl. No.: 18/958,694

(22) Filed: Nov. 25, 2024

Related U.S. Application Data
(60) Provisional application No. 63/603,573, filed on Nov. 28, 2023.
(60) Provisional application No. 63/603,578, filed on Nov. 28, 2023.

(51) Int. Cl. A61B 34/30; A61B 1/005

(52) U.S. Cl. CPC A61B 34/30; A61B 1/0057; A61B 2034/301

(57) ABSTRACT

Disclosed are a robotic apparatus, a catheter and method for use, with a push-pull assembly that includes a pusher hypotube with a distal end extending towards the continuum robot, a proximal end extending towards the controller, and a hollow extending through at least a part of a longitudinal length thereof; a support sleeve with a proximal end slidably maintained within the hollow; and a driving wire with a proximal end affixed to the pusher hypotube and a portion extending through the support sleeve. A deformable member may be positioned within a portion of the hollow that surrounds at least a portion of the driving wire, with the deformable member maintaining at least one of a uniform inner diameter and/or a uniform outer diameter when compressed. The deformable member may be formed of metal with a predetermined pattern that surrounds at least at part of the hub hypotube.

### CROSS-REFERENCE TO RELATED APPLICATION(S)

This application claims the priority under 35 U.S.C. § 119 (e) to U.S. Provisional Application No. 63/603,573 (Docket 2600-30863-prov) and to U.S. Provisional Application No. 63/603,578 (Docket 2600-30912-prov), each of which were filed on Nov. 28, 2023, the entire disclosure of each of which is incorporated herein by reference.

### FIELD OF THE DISCLOSURE

The present disclosure relates generally to medical devices and, more particularly to a continuum robot (also referred to as snake) applicable to guide interventional tools and instruments, such as endoscopes and other tools, in medical procedures.

### BACKGROUND OF THE DISCLOSURE

A continuum robot includes a plurality of bending sections having a flexible structure, wherein the shape of the continuum robot is controlled by deforming the bending sections. The snake has two significant advantages over existing robots including rigid links. The first advantage is that the snake can move along a curve in a narrow space or in an environment with scattered objects in which the rigid link robot may get stuck. The second advantage is that it is possible to operate the snake without damaging surrounding fragile elements because the snake has intrinsic flexibility.

In recent years, minimally invasive medical care, with which burden on the patient can be reduced and quality of life (QOL) after treatment or inspection can be improved, has been attracting attention. A surgery or inspection using an endoscope is a typical example of minimally invasive medical care. For example, a laparoscopic surgery is advantageous over a conventional abdominal surgery in that it can be performed with a smaller surgical wound, which results in a shorter stay in the hospital and less damage to the appearance.

Endoscopes used for the minimally invasive medical care are roughly divided into rigid endoscopes and soft endoscopes. With a rigid endoscope, although clear images can be obtained, the direction in which an observation target can be observed is limited. In addition, when the rigid endoscope is inserted into a curved organ, such as the esophagus, large intestine, or urethra, an insertion portion of the rigid endoscope presses the organ and causes pain for the patient. In contrast, a soft endoscope includes an insertion portion formed of a bendable member, so that a large area can be observed in detail by adjusting the bending angle of the distal end of the endoscope. In addition, by bending the insertion portion along an insertion path, burden on the patient can be reduced. When the number of bendable portions is increased, the endoscope can be inserted to a deep area of the body without causing the endoscope to come into contact with tissue even when the insertion path has a complex curved shape.

Accordingly, soft endoscopes having a plurality of bendable portions have been researched and developed.

Various related art disclosures in the field include U.S. Pat. No. 11,559,190, which discusses a steerable device with push-pull actuators and breakout unit, as well as WO 2022/146751 which discusses a steerable snake with push-pull rod structure. U.S. 2022/0202277 A1 discusses a medical apparatus having a bendable body with a driving wire; a break-out wire attached to the driving wire, with a distal end of the break-out wire attached to a proximal end of the driving wire; a distal guide tube guiding the driving wire and ending before the break-out wire with a space; a resilient element abutting the driving wire along at least a portion of a longitudinal direction of the driving wire; and an actuator configured to retract and advance the driving wire via the break-out wire thereby maneuvering the bendable body. Each of the afore-mentioned disclosures are incorporated herein by reference.

When controlling the bendable medical device by pushing or pulling the small diameter drive wires, the amount of operating force that can be applied to the drive wires is limited by the critical buckling force of the specific wire diameter and material. For the bendable medical device, using small diameter wires is unavoidable due to space constraints from the target anatomy, tool dimensions, etc. To prevent wire buckling, continuous support may be provided around the drive wires throughout the entire length of the bendable medical device.

It is difficult to fully support a small unit during assembly, with assembly restrictions including: Push-ability, which translates the push/pull motion from an actuator to the catheter, and adds stiffness/support to the drive wires and hypotubes to be pushed and pulled without buckling or deformation; Clamp strength, which allows the wire end to be attached/detached to the actuator clamp with high tensile strength; Alignment, which positions/guides the drive wires from the actuator clamps up to the catheter lumens; Durability/catheter robustness, regarding which the assembly should be able to withstand repeated clamping, pushing and pulling, and catheter handling without damage; and Manufacturing cost, preferably with a minimized number of parts and assembly steps, to obtain a catheter that is small in size.

As such, a need exists for further refinement of bendable medical devices and continuum robots, to reduce/eliminate buckling of the bendable body, and related limitations.

### SUMMARY

To address such exemplary needs in the industry, an aspect of the present disclosure provides a push-pull assembly for operably connecting a continuum robot with a controller that includes a pusher hypotube with a distal end extending towards the continuum robot, a proximal end extending towards the controller, and a hollow extending through at least a part of a longitudinal length thereof; a support sleeve with a proximal end slidably maintained within the hollow of the pusher hypotube; and a driving wire with a proximal end affixed to the pusher hypotube and a portion extending through the support sleeve.

Another aspect of the present disclosure provides a robotic apparatus that includes a continuum robot including a driving wire and a distal section configured to change a posture and/or a pose in response to driving of the driving wire by a controller; a hub body; a pusher hypotube; and a support sleeve extending at least partially through or across the hub body. Between the controller and the hub body, the driving wire is surrounded and supported by at least one of the support sleeve and the pusher hypotube.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present innovation will become apparent from the following detailed description when taken in conjunction with the accompanying figures showing illustrative embodiments of the present innovation.

FIG. 1 is a block diagram of an exemplary medial system including ancillary components and a bendable medical device.

FIG. 2 illustrates components of a continuum robot.

FIG. 3 illustrates relative connections between an actuator and catheter of a steerable robotic catheter.

FIG. 4 is a cut away view of a continuum robot catheter shaft.

FIG. 5 illustrates a clamp mechanism.

FIG. 6 illustrates unsupported sections of a plurality of pusher rods.

FIG. 7 illustrates a reinforced section of a pusher rod.

FIG. 8 illustrates damage to the unsupported section of pusher rods of a catheter.

FIG. 9 illustrates buckling of a hub hypotube.

FIG. 10 is a cross section of actuator clamps according to the present disclosure.

FIG. 11 is a perspective view of a miniaturized actuator and guide tubes according to the present disclosure.

FIG. 12 is a profile view of a push-pull assembly, according to the present disclosure.

FIGS. 13A to 13C are profile views of the push-pull assembly illustrating movement during a push operation and a pull operation, according to the present disclosure.

FIGS. 14A to 14B are profile views illustrating movement during a push operation and a pull operation of the reinforced section of the pusher rod of FIG. 7.

FIG. 15A is a perspective view of a hub assembly and push-pull assembly.

FIG. 15B is a top view of a hub assembly and push-pull assembly, according to the present disclosure.

FIG. 16 is a cutaway profile view of a single-part extruded hub body, according to the present disclosure.

FIG. 17 is a rear perspective view of the hub body illustrating the single part hub body having multiple channels, according to the present disclosure.

FIG. 18 is a cutaway view of the hub body illustrating a single channel of the hub body, according to the present disclosure.

FIG. 19 is a cutaway profile view of the hub body, according to the present disclosure.

FIG. 20 is a partial cutaway front perspective view of the hub body, according to the present disclosure.

FIG. 21 illustrates a curved path of the hub hypotubes and the hub cone, according to the present disclosure.

FIG. 22 illustrates an uninsulated clamp rod.

FIG. 23 is a cutaway profile view of the hub body with the hub insert tube, according to the present disclosure.

FIG. 24 is a rear profile view of an elastomeric cone cover being slid over a hub cone, according to the present disclosure.

FIG. 25 is a cutaway profile view of a cone cover being compressed by a catheter outer shell, according to the present disclosure.

FIG. 26 is a cutaway profile of a clamp rod assembly, according to the present disclosure.

FIG. 27 is a profile view of a hypotube sleeve with actuator guide tube, according to the present disclosure.

FIG. 28 illustrates an internal arrangement of the hypotube sleeve inserted into the actuator.

FIG. 29 is a cutaway longitudinal view illustrating non-circular profiles of clamp rod.

FIG. 30 is a profile view of a connection between a catheter and hub with exposed drive wires.

FIG. 31 is a profile view of a pusher hypotube with selective crimps according to the present disclosure.

FIG. 32 is a profile view of catheter to hub connection, according to the present disclosure.

FIG. 33 illustrates an assembly pre-reflow condition, according to the present disclosure.

FIG. 34 shows the assembly in a reflowed condition, according to the present disclosure.

FIG. 35 is a profile view of a catheter, according to the present disclosure.

FIG. 36 is a perspective view of a catheter assembly, according to the present disclosure

FIG. 37 is a profile cutaway view of a hub support spring, according to the present disclosure.

FIG. 38A to 38C illustrate a push-pull assembly with the hub support spring, according to the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

In the subject disclosure, systems and mechanisms of a continuum robot are described, followed by continuum robot support elements for reducing buckling, as well as the systems and procedures associated with the continuum robot and said support elements.

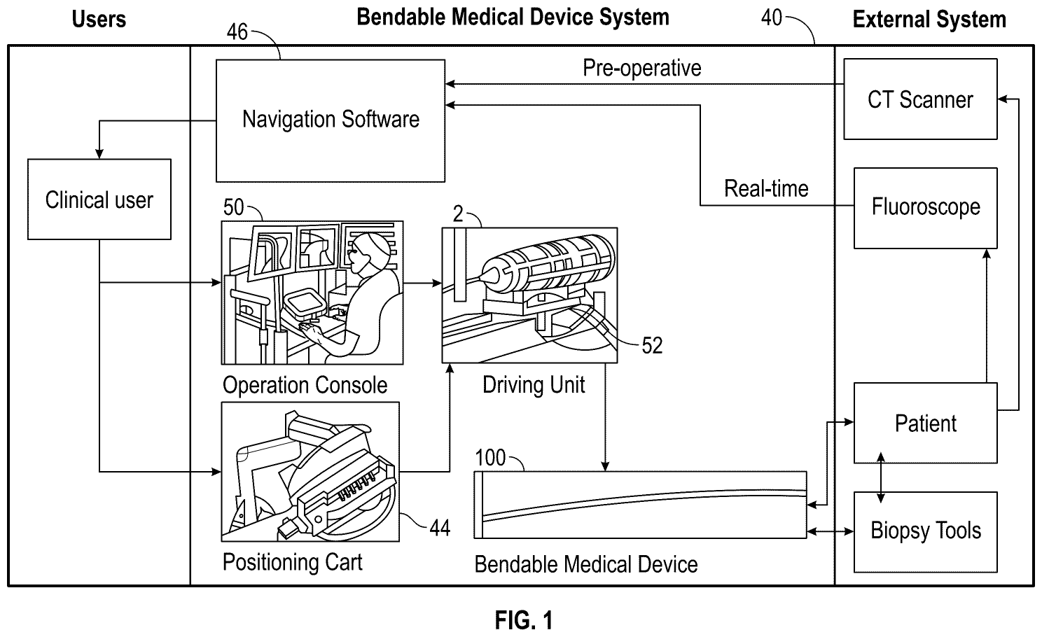

FIG. 1 is a block diagram of an exemplary medial system including ancillary components and a bendable medical device.

As illustrated in FIG. 1, the system 40 includes an actuator or driving unit 2 (also referred to as driver) for driving the drive wires, and having a base stage 52, a bendable medical device 100 (also referred to as a steerable catheter), a positioning cart 44, an operation console 50, having push-button, thumb-stick, and/or joystick, and navigation software 46. The medical device system 40 is capable of interacting with external system component and clinical users to facilitate use in a patient.

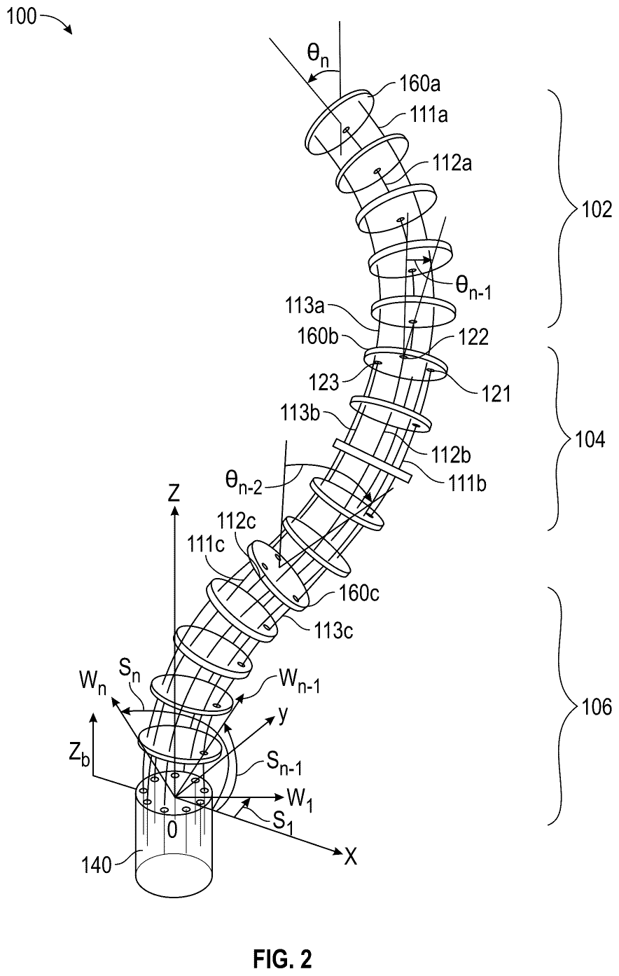

FIG. 2 illustrates components of a continuum robot.

As shown in FIG. 2, the continuum robot 100, comprises drive wires 111b, 112b and 113b, which are connected to connection portions 121, 122 and 123, respectively, found on an end disc 160b, for controlling the middle bending section 104. Additional drive wires (three for each of the other bendable sections 102 and 106) 111a, 111c, 112a, 112c, 113a, 113c, are attached at the distal ends of each bendable section 102 and 106, to the respective end disc 160a and 160c.

Each bending section is operated similarly. Thus, the focus herein on one bending section, i.e., the middle bending section 104, will be recognized to apply to the other sections. The posture of the bending section 104 is controlled by pushing and pulling the wires 111b to 113b by using at least one actuator or by manual operation.

Moreover, the continuum robot 100 attaches to a catheter shaft 140, which may be disposed on a base stage 52 (FIG. 1) and can be moved by the base stage 52 in the longitudinal direction. Thus, it is possible to advance and retard the robot 1 into a target structure by advancing and retarding the base stage 52.

An operational console 50 (FIG. 1) may indicate a driving amount to the base stage 52 and, independently, to the actuator 2 or controller. Herein, the operational console 50 may also be described or eluded to as a control system. The operational console 50 may include dedicated hardware including a field-programmable gate array (FPGA) and the like; and/or may be a computer including a storage unit, a work memory, and a central processing unit (CPU). In the case where the operational console 50 is a computer, the storage unit may store a software program corresponding to a control system algorithm and the CPU may expand the program in the work memory, and may execute the program line by line, for the computer to function as the operational console 50. In either case, the operational console 50 may communicably connect with the base stage 52 and the actuator 2, and the operational console 50 may send signals representing the driving amount and configuration to these control targets, which may be imputed by an end user through push buttons, joystick or the like.

With respect to FIG. 2, the definitions of symbols are as follows: l_{d}=the length of the central axis a bending section; θₙ=the bending angle of the distal end; ζₙ=the rotational angle of the distal end; ρₙ=the radius of curvature of a bending section.

At detailed above, the continuum robot 100 includes at least one distal bending section 102 with robotic insertion and removal of the continuum robot 100 from the target.

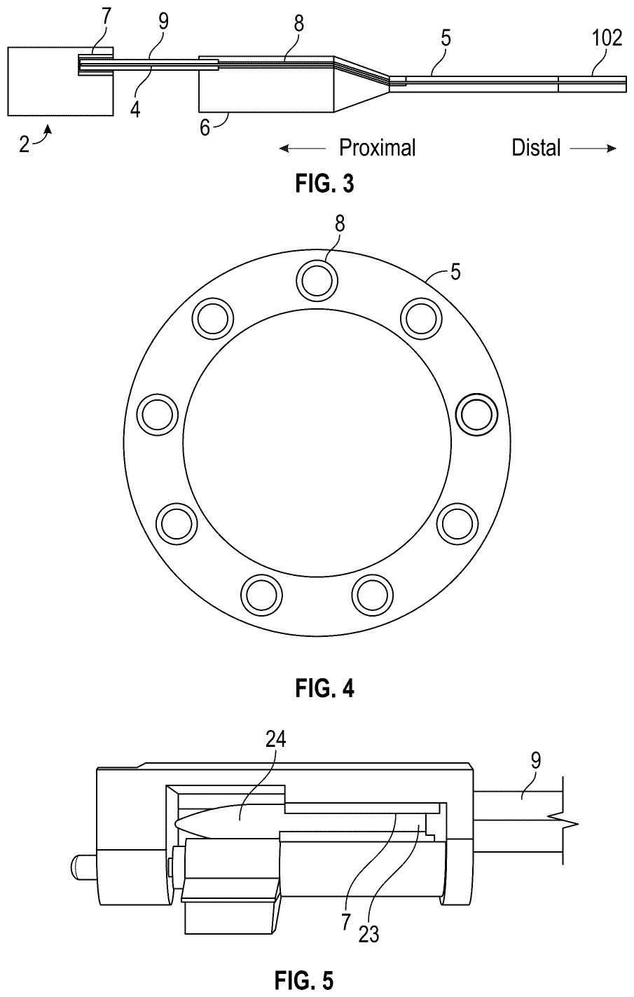

FIG. 3 illustrates relative connections between an actuator and catheter of a steerable robotic catheter.

The steerable robotic catheter 100 may be controlled by push/pull drive wires 3 and may be used for lung biopsies, medical procedures, and similar operations.

The catheter shaft 5 has at least one distal bending section 102, with at least three drive wires 4 terminating in each section to control bend angle and plane. An actuator 2 may selectively push/pull drive wires 4 to control the distal bending section 102. Pusher rods 9 on the catheter are clamped by an actuator clamp 7 on the actuator 2. Pusher rods 9 may be attached/detached from respective clamps 7. Pusher rods 9 are fixedly attached to the proximal end of respective driving wires 4. The drive wires 4 may slide along respective hub hypotubes 8, also referred to as support sleeves, which are anchored at distal ends of the hub body 6, and slide into the catheter shaft 5.

The hub body 6 may have a straight section at the proximal end, and a pitch diameter transition at the distal end to reduce the wire pitch diameter from that of the actuator clamps 7 down to the lumen pitch diameter in the catheter shaft 5. The push/pull mechanism is supported by the straight portion of the hub body 6.

For a small diameter catheter, the hub hypotubes will have correspondingly small diameters for connection to the catheter shaft, with pusher rods of the same size as the hub hypotubes. While this structure may support the drive wires, the small hypotubes require reinforcement to achieve the push-ability, clamp strength, and durability for operation of the push-pull assembly.

FIG. 4 is a cut away view of a continuum robot catheter shaft.

As illustrated in FIG. 4, the catheter shaft 5 may have a central tool channel and nine drive wires, supported by 26TW hub hypotubes having a 0.012″ inner diameter (ID) and an outer diameter (OD) of 0.018″. Limited space exists to increase the hub hypotube diameter. Challenges using small diameter catheters include insufficient clamp strength. The small diameter available at the clamped end of the pusher rod fails to provide sufficient surface area and/or rigidity to achieve a high clamp strength to transmit push/pull force from the actuator.

FIG. 5 illustrates a clamp mechanism.

As illustrated in FIG. 5, a hypotube clamp sleeve 24 of a clamp rod 23 attached to the pusher rod 9 may attach to an actuator clamp 7, and the hypotube clamp sleeve 24 may be a sheet metal clip. The mechanism of the clamp rod 23 may require a 3 mm clamped diameter, making it difficult to compress smaller hypotubes without hypotube bending/damage. Conventionally, actuator clamps may use a set screw to tighten against the clamped end, which requires a larger clamp diameter to achieve a sufficient set screw contact area.

Limited push-ability may also be a concern. If the actuator is spaced apart from the hub body, a small diameter pusher rod may buckle in response to actuator pushing force without. For example, a 26TW hypotube may buckle with less than 5 N, if unsupported for more than 50 mm. Lack of durability/robustness may also be a concern. A pusher hypotube may be damaged by external sources, e.g., during assembly, transport, general handling. An outer shell of the catheter covers the hub body. However, the pusher hypotubes may be exposed to facilitate loading into the actuator clamps. Bending/kinking may cause permanent damage to small hypotubes. The drive wire cannot be pushed once the pusher hypotube is bent and the hypotube cannot be restored to a perfectly straight condition, as needed for proper operation. The pusher hypotube may be reinforced to improve stiffness and buckling resistance. Since clearance is needed to push forward by at least one push stroke length without interference, a reinforcing tube may not cover the pusher hypotube at the section that is proximal to the hub body. The location of the transition from the pusher hypotube to the clamp rod may also create a weak point for buckling/damage since this area is exposed/unsupported.

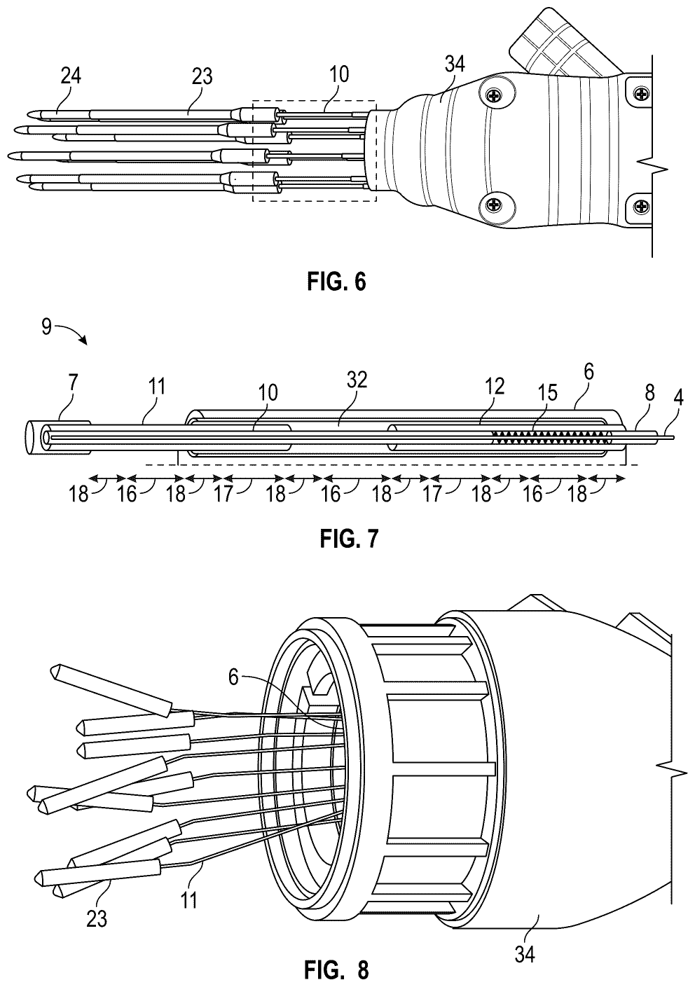

FIG. 6 illustrates unsupported sections of a plurality of pusher rods. The structure illustrated in FIG. 6 is used for a small diameter continuum robot to reinforce the pusher hypotube 10 when a transition area allows for support by the hub body, to avoid damage to the pusher hypotube 10 based on forces exerted by the hypotube clamp sleeves 24 and clamp rods 23, as well as other sources of damage.

FIG. 7 illustrates a reinforced section of a pusher rod.

In the reinforced section of a pusher rod illustrated in FIG. 7, a pusher reinforcement hypotube 11 adds stiffness to the pusher hypotube 10. As illustrated in FIG. 7, drive wire 4 extends through hub hypotube 8 and the hub hypotube 8 extends through hub body 6 within outer guiding hypotube 12, which includes a resilient member, e.g., a compression spring, 15, one end of which applies a force on pusher hypotube 10.

A pusher reinforcement hypotube 11 surrounds a portion of the pusher hypotube 10 opposite an end of the pusher reinforcement hypotube 11 that attaches to the clamp rod 23. As illustrated in FIG. 7, a hub guide channel 32 aligns the pusher reinforcement hypotube 11 with the outer guiding hypotube 12. This arrangement of stacked hypotubes improves push-ability, clamp strength, and durability/robustness of the pusher rod 9. However, downsides may exist in this reinforced of the pusher rod 9.

A reduced diameter pusher rod is a downside that may exist in the reinforced pusher rod of FIG. 7, and the pusher reinforcement hypotube may not be sufficiently large to provide stiffness, clamp strength, and durability. Metal hypotubes are ideal for push-pull assembly components, as they are low-cost, tightly toleranced, thin-walled parts with low friction. Low spring friction is necessary for the outer guiding hypotube 12, and the pusher reinforcement hypotube 11 needs to be the same size as the outer guiding hypotube 12. However, the hypotube sizes available may not be sufficiently large for clamping or may not be supported outside the hub without damage. For example, the largest standard hypotube size to fit closely to the 26TW hypotube (0.018″ ID) is 20HV, which has a 0.0355″/0.90 mm outer diameter. The actual catheter uses 21RW hypotubes, which have a 0.032″/0.81 mm outer diameter. The stacked 21RW/26TW hypotubes are small, and may buckle with less than 20 N pushing force if the distance to the actuator is greater than 100 mm. A separate clamp rod 23 may be added to the proximal end of the pusher rod, to add reinforcement/buckling support. However, a section proximal to the hub body remains where the pusher reinforcement hypotube cannot be reinforced and may still be damaged.

FIG. 8 illustrates damage to the unsupported section of pusher rods of a catheter.

As illustrated in FIG. 8, hub body 6 include a catheter outer shell 34 and pusher reinforcement hypotubes 11, with clamp rods on proximal ends thereof, extending from the hub body 6. FIG. 8 illustrates damage to the pusher reinforcement hypotubes 11 of the unsupported section of the pusher rods.

More complex manufacturing, including a greater number of parts with associated increased complexity of assembly, is a another downside that may exist in the reinforced of the pusher rod of FIG. 7. Adding parts and assembly steps increases the cost of materials/labor. For a catheter with multiple drive wires, the cost of the push-pull assembly is multiplied and can contribute significantly to the total catheter cost. Also, hypotube bonding may be needed when multiple hypotubes must be bonded together into assemblies. Hypotube bonding is difficult and/or expensive to bond small hypotubes accurately with high bond strength. The stacked hypotube assembly of FIG. 7 has two hypotube assemblies with three total bonding locations. Adhesives may need to be used to bond hypotube assemblies. However, adhesive may not consistently provide sufficient bond strength and may cause other failures, e.g., wicking into hypotubes and springs, adding wire friction or blocking push stroke, etc. Laser welding may be used to bond hypotubes with high bond strength, accuracy, and repeatability. However, laser welding is expensive, especially for high volume manufacturing. Also, increased length of the stacked hypotube assemblies have two overlapping sections which need clearances on both sides for the push and pull stroke, which may almost double the length of the hub body.

Thus, as discussed herein, various devices, methods and systems are provided to overcome the above shortcomings of conventional systems.

A first aspect of the present disclosure provides a push-pull assembly for a small diameter robotic catheter which accomplishes the same functionality as the stacked hypotube structure that minimizes the number of hypotubes, assembly steps, and assembly length.

In addition to supporting the push-pull assembly, the hub body aligns components of the push-pull assembly from the actuator to the catheter, creating a path for tools to be inserted through the catheter via the tool channel. For a small diameter catheter, it may be difficult to provide the necessary alignment and support to prevent buckling if the push-pull assembly components are curved/bent to accommodate the pitch diameter transition and tool channel.

The catheter may have a central tool channel through the entire working length, allowing physicians to utilize biopsy tools and endoscopes during operations. Tools are inserted through the hub body and slide into the catheter's tool channel. An exit port of the tool channel needs to be accessible from the outside of the hub body, which may require increasing the pitch diameter to create space between drive wires.

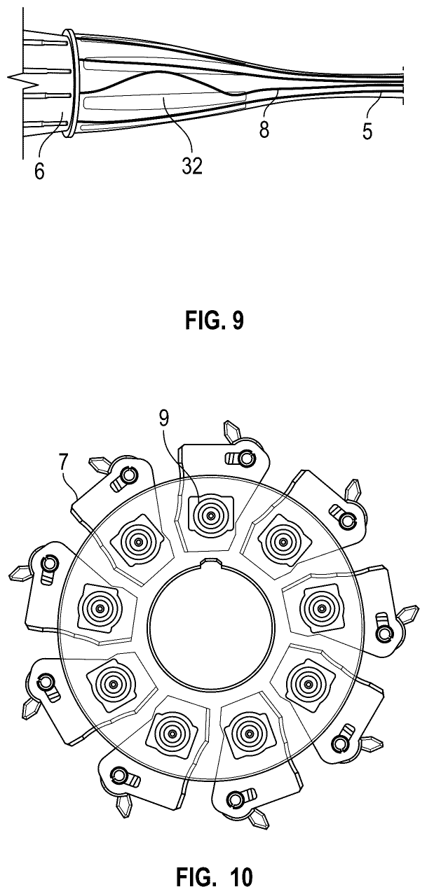

FIG. 9 illustrates buckling of a hub hypotube.

The hub hypotube 8 illustrated in FIG. 9 is not fully supported at a pitch diameter transition, and buckling has resulted when upon pushing of the hub hypotube 8. Thus, hub hypotubes 8 require support in the curved section to prevent outward buckling during pushing operation.

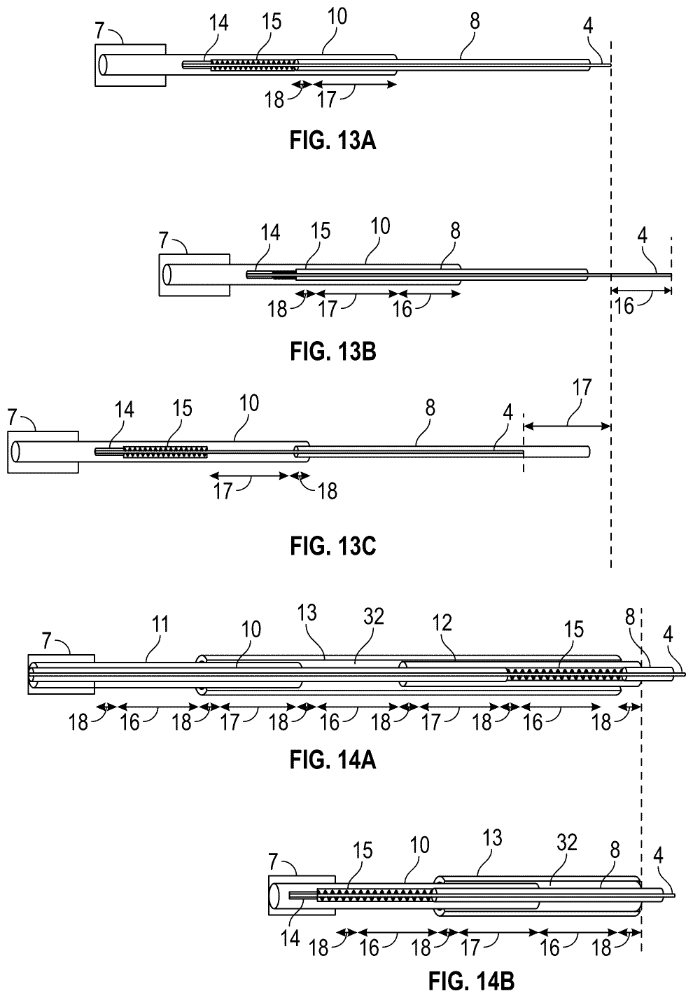

Hub body 6 may provide support for curved hypotubes to reduce buckling. Hub body 6 may include multiple hub guide channels 32 for supporting the push-pull assembly (FIGS. 12 to 13C). Thus, the catheter 1 may have a pitch diameter reduction between the actuator 2 and catheter shaft 5, with hub guide channels 32 created as a single part with multiple channels, or as multiple single-lumen parts.

FIG. 10 is a cross section view of actuator clamps of the present disclosure.

The actuator clamp illustrated in FIG. 10 has nine evenly spaced drive wires, and an actuator pitch diameter may be 22 mm, with pusher rods of 3 mm diameter. Reduction of actuator pitch diameter is limited by the clamp dimensions, which are already minimized and require clearances to freely slide. Due to the minimum size and spacing of the actuator components of a small diameter catheter, portions of the drive wires that are releasably connected to the actuator may be pushed/pulled at a larger pitch diameter than portions of the wires that are located in the catheter shaft. Thus, an aspect of the present disclosure provides a larger actuator pitch diameter.

Accommodation of the pitch diameter transition presents challenges that include alignment of the push-pull assembly, and the hub body may need to align the pusher rod 9 to the actuator clamp 7, which is positioned at a proximal end of the assembled hub body. Also, the hub hypotube 8 may be aligned to the catheter shaft lumen at the distal end of the hub body. The pusher rod 9 and hub hypotube 9 also need to be positioned substantially parallel to the catheter shaft and the direction in which the actuator pushing/pulling.

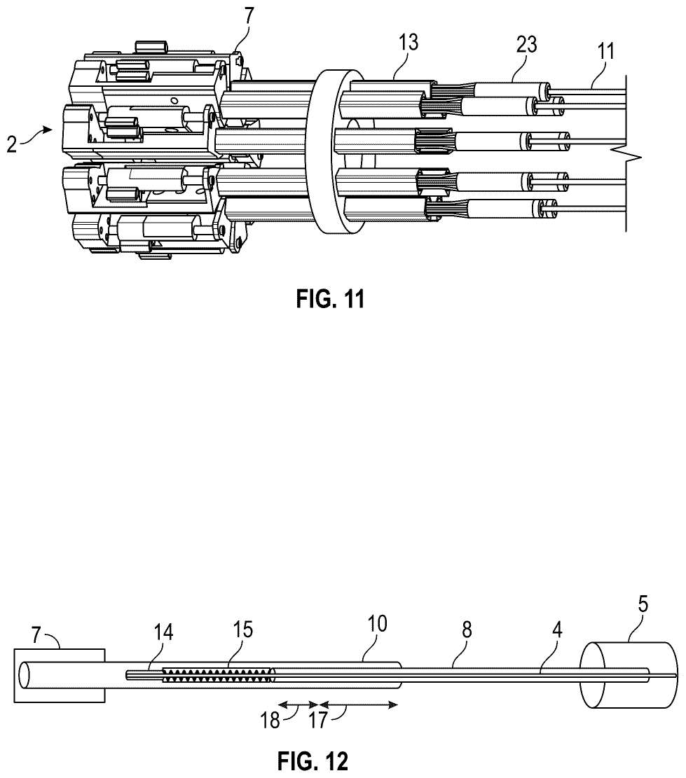

FIG. 11 is a perspective view of a miniaturized actuator and guide tubes.

The actuator of FIG. 11 includes sheet metal driven by a cam gear to compressably clamp and lock a respective clamp rod 23 circumference. Since the clamp mechanism does not require perfect alignment of each clamp rod 23, such clamp mechanism is convenient for expedited alignment and fixing multiple wires of a catheter. The guide tubes surround the clamp rods 23 and slide through a front plate to maintain concentricity of the clamp rods 23 with the actuator. This configuration allows for miniaturization of the actuator.

The clamp mechanism provides a metal clamped end and electrical isolation. The clamp rods 23 need to have a uniform and tightly toleranced outer diameter to achieve high clamp strength with this clamp. The clamp rods 23 also need to be sufficiently durable to withstand repeated clamping without deformation. Typical insulating materials such as plastics or ceramics can be easily deformed/damaged, or require expensive molds to achieve tight tolerances. A clamp rod tube fully covers the pusher hypotube 10 and is clamped by the actuator clamps 7.

FIG. 12 is a profile view of a push-pull assembly, according to the present disclosure.

As illustrated in FIG. 12, the push-pull assembly includes a push-pull drive wire 4, a pusher hypotube 10, a hub hypotube 8, and a compression spring 15. The pusher hypotube 10 is at least one of affixed to and/or adjacent to the actuator clamp 7, and a proximal end of the drive wire 4 is affixed to the pusher hypotube 10 within an interior recess of the pusher hypotube 10. A proximal end of compression spring 15 is adjacent to a location where drive wire 4 is affixed within the pusher reinforcement hypotube 10. A distal end of compression spring 15 abuts a proximal end of the hub hypotube 8, with the drive wire 4 extending substantially through centers of the pusher reinforcement hypotube 10 and the hub hypotube 8, into the catheter shaft 5. The hub hypotube 8 fully guides/supports the drive wire 4 up to the catheter shaft 5, and the pusher hypotube 10 may be bonded to the proximal end of the drive wire 4.

FIGS. 13A to 13C are profile views of the push-pull assembly illustrating movement during a push operation and a pull operation, according to the present disclosure.

FIG. 13A corresponds to FIG. 12, in which the push-pull assembly is in a neutral mode. For clarity in highlighting the location of point P along the drive wire 4 during the operations of the push-pull assembly, FIGS. 13A to 13C omit the catheter shaft 5. In the following description, the distal end of the drive wire 4 is covered by at least one of the hub hypotube and the catheter shaft 5.

In FIG. 13B, the push-pull assembly is in an extended mode, in which the actuator applies a pushing force on the actuator clamp 7 and on the distal end of the pusher hypotube 10, thereby compressing compression spring 15 while moving the drive wire 4 outward, to move point P of drive wire 4 closer to the patient P on which a procedure is being performed by a maximum push stroke length 16.

In FIG. 13C, the push-pull assembly is in a retracted mode, in which the actuator applies a pulling force on the actuator clamp 7 and on the distal end of the pusher hypotube 10, thereby allowing the compression spring 15 to return to a neutral position, with a proximal end of the hub hypotube 8 moving away from a distal end of the compression spring 15 by a first distance, with a distal end of the hub hypotube 8 remaining extended at least one of through or across the hub body 6 by the first distance, thereby maintaining a protective cover on the push/pull wires 4. A maximum actuator force may be applied to the drive wire 4 as a force limit where the actuator/software disengages the catheter.

The pusher hypotube 10 fits closely over the hub hypotube 8, and a minimum hypotube overlap distance 18 is maintained to provide rigidity and to ensure alignment of the pusher rod 8 and hub hypotube 7 when the actuator is retracted by a maximum amount.

In FIG. 13B, the push-pull assembly is pushed forward to the maximum push stroke distance and, in FIG. 13C, the push-pull assembly is pulled back to the maximum pull stroke distance. That is, the actuator clamp 7 pushes/pulls on the pusher hypotube 9 to control a position of a distal end of the drive wire 4, thereby controlling the distal bending section 102 of the catheter.

A proximal end of the pusher hypotube 10 is clamped by the actuator clamp 7. A compression spring 15 covers the unsupported section of drive wire 3 inside the pusher hypotube 9, between the bonded end of the drive wire 3 and the proximal end of the hub hypotube 7. The ID of the compression spring 15 fits over the drive wires 4, and the OD of the spring 15 fits inside the pusher hypotube ID 9. The difference between a free length and a solid length of the compression spring 15 is at least one push stroke length 16. The compression spring 15 may have closed ends, for the hub hypotube 8 to push on a flat surface.

By way of non-limiting example, the dimensions of the movements illustrated in FIGS. 13A to 13C include a maximum push stroke 16 and pull stroke 17 lengths both that may be 16 mm; and a minimum hypotube overlap 18 that may be 5 mm. The minimum hypotube overlap 18 includes tolerances for actuator/software hard stops and assembly, to ensure that no collisions occur in the case there is control lag. The drive wires 4 may be 0.0095″ nitinol wires. The pusher hypotube 10 may be 304SS 21RW hypotube, 0.020″ ID/0.032″ OD, 61 mm length. The hub hypotube 8 may be a 304SS 26TW hypotube, 0.012″ ID/0.018″ OD, 126 mm length. The compression spring may be 15 is 0.0025″ diameter nitinol wire, 0.018″ OD/0.013″ ID; and 140 active coils, with a free length of 30 mm and solid length of 9 mm (to achieve a 21 mm clearance for 16 mm push stroke+5 mm safety margins).

Thus, advantages are provided that include prevention of wire/spring buckling during pushing, with the configuration of FIGS. 12 to 13C preventing drive wire 4 and spring 15 from buckling outwards when pushed. The spring 15 supports the wire from buckling when pushed, and the spring length compresses as the pusher rod is moved forward. The pusher hypotube 10 prevents the spring from buckling.

The configuration of FIGS. 12 to 13C also provides alignment, with the pusher hypotube overlap 18 ensuring that the pusher hypotube 10 and hub hypotube 8 remain fully aligned at the maximum push and pull stroke, with a minimum overlap being maintained that provided rigidity. Pusher rod durability/robustness is also provided, with the larger diameter of the pusher hypotube 10 better resisting bending and damage, and with the uniform diameter outside the hub body 6 eliminating unsupported transitions/weak sections. Also, fewer hypotubes are utilized, with FIGS. 12 to 13C using only two hypotubes, rather than three hypotubes, with a stacked hypotube version for a small diameter catheter using five hypotubes. Fewer hypotubes in the push-pull assembly reduces cost and assembly steps. Also, no hypotube bonding is necessary. Since there is only one hypotube in the pusher rod 9 and one for the catheter connection, no hypotube bonding is required. Hypotube bonding is expensive, especially for multiple drive wires. Additionally, bonded parts create potential failure locations. A shorter assembly length is provided for a small catheter, with the push-pull assembly in the arrangement of FIGS. 12 to 13C being much shorter than in other systems, where the stacked hypotubes add multiple overlap lengths. Smaller size is desirable for catheter manufacturability.

FIGS. 14A to 14B are profile views illustrating movement during a push operation and a pull operation of the reinforced section of a pusher rod of FIG. 7.

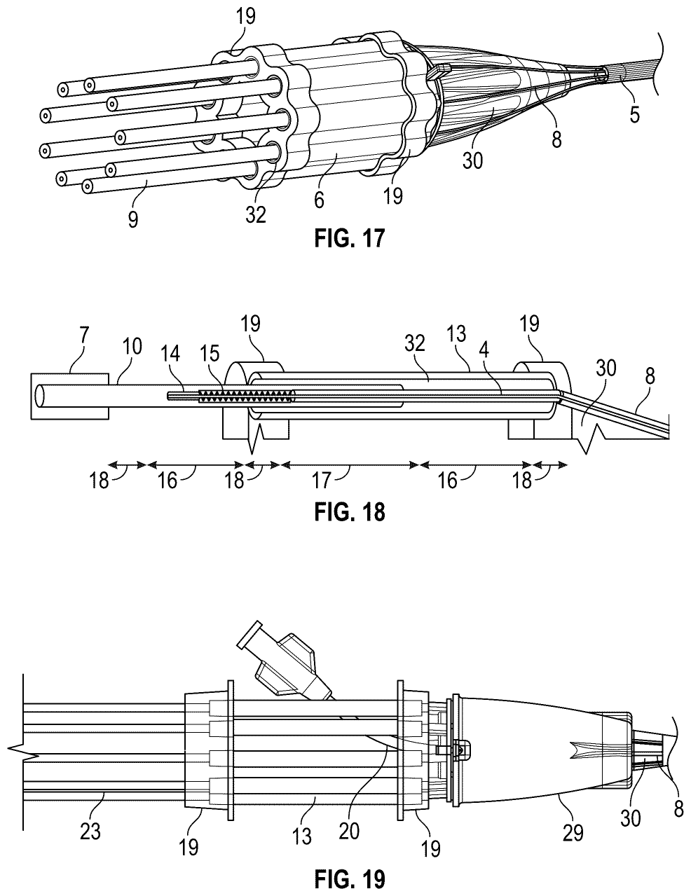

The push-pull assembly of FIGS. 14A and 17B corresponds to the reinforced section of a pusher rod of FIG. 7. For conciseness, details of the pusher rod of FIG. 7 are incorporated herein.

Comparison of FIGS. 13A-13C with FIGS. 17A-FIG. 17B illustrates, inter alia, the shorter assembly length provided by the push-pull assembly of the present disclosure.

Accordingly, the present disclosure provides a robotic apparatus that includes an actuator 2, a continuum robot 100, a hub body 6, and a support sleeve. The actuator 2 may drive at least one driving wire 4 and the actuator 2 includes an actuator clamp 7 that is configured to drive a proximal end of the at least one driving wire 4. The continuum robot 100 may include a proximal section and a distal section, which is configured to bend in response the actuator 2 driving the at least one driving wire 4. The hub body 6 may be affixed to the continuum robot 100, and the hub body 6 may connect a proximal end of the at least one driving wire 4 to the actuator 2. The support sleeve, i.e., hub hypotube 8, may support at least a portion of the at least one driving wire 4 between the actuator 2 and the hub body 6. The support sleeve may at least partially extend at least one of through or across the hub body 6 and may surround the at least one driving wire 4 between the actuator 2 and the hub body 6.

The robotic apparatus may also include a pusher hypotube 10 with a resilient member 15 therein. A proximal end of the pusher hypotube 10 may be fixed to the actuator clamp 7 and the support sleeve may guide and support the at least one drive wire 4 from a catheter shaft 5 past a distal end of the pusher hypotube 10. A proximal end of the support sleeve may extend into the distal end of the pusher hypotube 10 and contact a distal end of the resilient member 15, and operation of the resilient member 15 may adjust an amount that the proximal end of the support sleeve (hub hypotube 8) extends into the distal end of the pusher hypotube 10.

The robotic apparatus may include a pusher hypotube 10 with a resilient member 15 therein, which is coiled around a portion of the at least one driving wire 4. A proximal end of the at least one driving wire 4 may be affixed to the pusher hypotube 10 adjacent to a proximal end of the resilient member 15.

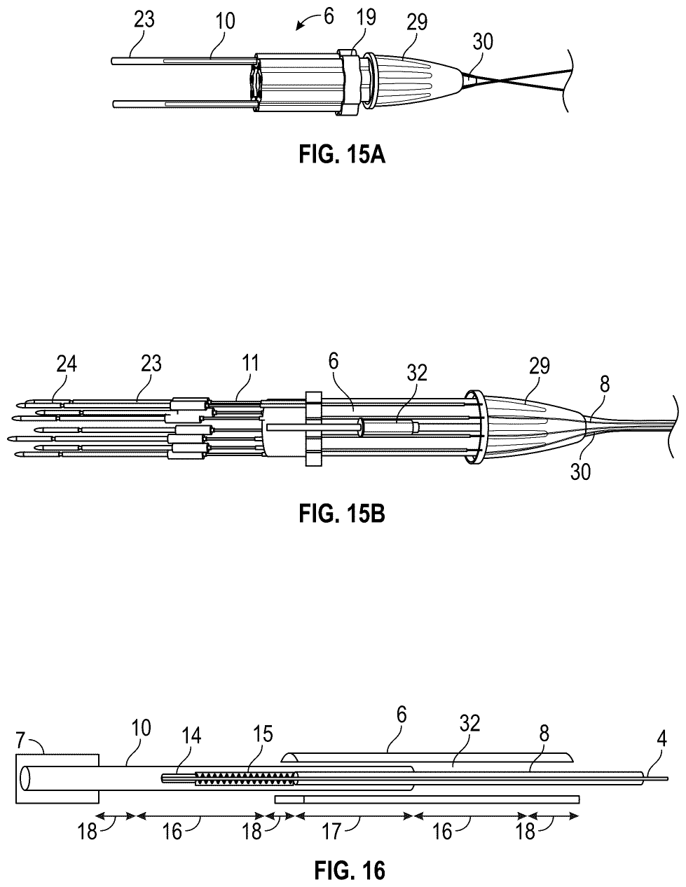

FIG. 15A is a perspective view of a hub assembly and push-pull assembly. FIG. 15B is a top view of a hub assembly and push-pull assembly according to the present disclosure.

FIG. 15A illustrates a structure of hub body 6, a cone cover 29 and hub cone 20 of the hub body 6, illustrating pitch diameter transition for the catheter and tool channel. As illustrated in FIGS. 15A and 15B, hub hypotubes 8 are curved at the location where the pitch diameter transition occurs, which increases the risk of buckling. Comparison of FIG. 15A with FIG. 15B illustrates, inter alia, the shorter assembly length provided by the push-pull assembly of the present disclosure.

FIG. 16 is a cutaway profile view of a single-part extruded hub body, according to the present disclosure. FIG. 17 is a rear perspective view of the hub body illustrating the single part hub body having multiple channels, according to the present disclosure.

The hub body 6 of FIGS. 16 and 17 is a single extruded part with a straight hub channel, as described herein. As shown in FIG. 17, hub guide disks 19 are provided at proximal and distal ends of the hub body 6. The hub disc at the distal end may be used for attachment of an extruded part to the hub cone 30.

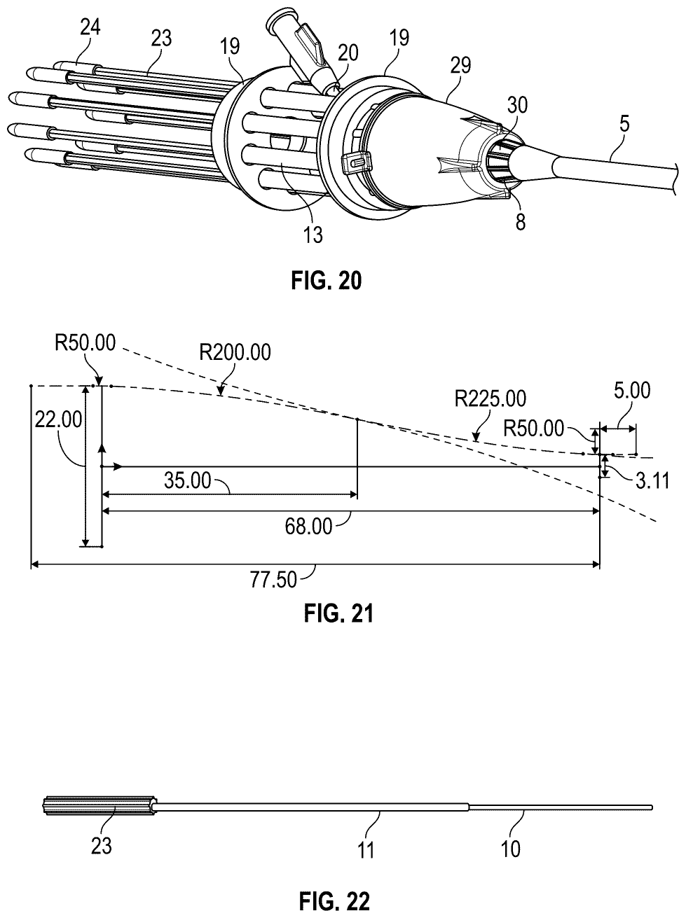

FIG. 18 is a cutaway view of the hub body illustrating a single channel of the hub body, according to the present disclosure. FIG. 19 is a cutaway profile view of the hub body, according to the present disclosure. FIG. 20 is a partial cutaway front perspective view of the hub body, according to the present disclosure.

As illustrated in FIGS. 18 to 20, the multi-part hub body may include a plurality of single lumen hub guide hypotubes 13 and guide disks 19, with the tool channel tube 20 exiting between the hub guide discs 19, with the catheter shaft 5 including a central lumen for tool passage through an entire working length thereof. The tool channel tube 20 may be inserted/bonded into the proximal end of the catheter shaft 5, to provide an inlet/outlet path for tools which are loaded from outside the hub body 6. An ID of the tool channel tube 20 may be sized to allow endoscopes and surgical tools to pass through without interference. An adapter, e.g., luer fitting, may be attached to the proximal end of the tool channel 20 outside the hub body 6 for connection to syringes, pumps and other instruments. The tool channel tube 20 may be constructed of material that is sufficiently flexible to bend along the exit path while maintaining resistance to buckling when tools are pushed therethrough.

FIG. 21 illustrates a curved path of the hub hypotubes and the hub cone, according to the present disclosure.

As illustrated in FIG. 21, within the hub cone 30, drive wires 4 undergo a transition from a pitch diameter on the actuator 2 side to a pitch diameter of the catheter 1. The catheter pitch diameter is smaller than the actuator pitch diameter and, to transition between the different pitches, the hub hypotubes 7 follow a curved path within the hub cone 29.

The hub cone 30 may attach to a distal end of the hub body 6. The hub hypotubes 8 may be bonded into grooves or channel features on the hub cone 30. The hub cone 30 curve starts at the larger actuator pitch diameter, reducing to the smaller catheter shaft pitch diameter. On a proximal end of hub body 6, the hub hypotubes 8 guide the drive wires 4 into straight parts of the hub guide channels 32. On a distal end of hub body 6, the hub hypotubes 8 guide the drive wires 4 into lumens of the catheter shaft 5.

The hub body 6 may be formed of a rigid material with hub guide channels 32 for respective drive wires 4. The hub body 6 may be a single part with multiple straight hub guide channels 32, or may have multiple single lumen parts. Proximal ends of the hub guide channels 32 are aligned at a same pitch diameter as the actuator clamps 6. The ID of the hub guide channel 32 is sized for the OD of the pusher hypotube 10, with clearance to allow the pusher hypotube 10 to easily slide. The proximal end of the hub guide channel 32 overlaps the pusher hypotube 10 by at least one pull stroke length 16, plus a minimum hypotube overlap length 18. The distal end of the hub guide channel 32 overlaps the proximal end of the hub hypotube 8 by a minimum overlap length. The hub guide channel 32 material, or lining of the hub channel, has low friction to permit longitudinal movement of the pusher rod 9.

The distance between the distal edge of the actuator clamp 7 and the proximal edge of the hub body 6 may be at least one push stroke length 16, to allow clearance for the pusher rod 9 to slide into the hub guide channel 32 without interference. For the multiple-part hub body 6, a hub guide channel 32 is created by multiple single lumen hub guide tubes, which may be extruded tubing. The hub guide tubes may be supported/positioned at the actuator pitch diameter by hub guide disks 19 located at opposite ends. To hold the assembly together, the hub guide disks 19 may be bonded to the hub guide hypotubes 13, with the distal hub guide disks 19 attached to the hub cone 30. The hub guide disks 19 may be held in place by outer protective shells 34, which cover the entire hub body 6 up to the proximal end of the catheter shaft 5.

By way of non-limiting example, the hub guide channels 32 may be 3.0 mm ID, sized for a 2.5 mm OD pusher rod 9. The hub guide channels 32 may be located around a 22 mm pitch diameter, parallel to the catheter shaft 5, to align to the 22 mm actuator clamp pitch diameter and straight push/pull alignment. A minimum hub channel length may be 42 mm, based on push and pull stroke lengths of 16 mm, and a minimum overlap 18 of 5 mm.

Regarding the multi-part hub body 6, hub guide hypotubes 13, and guide disks 19 illustrated in FIG. 18, the hub guide hypotubes 13 may be Delrin®, i.e., Polyoxymethylene, extruded tubing with similar dimensions as the hub guide channels 32, as described above, cut to the similar length(s), with a 4.5 mm OD. The hub guide disks 19 may be two-sided molded parts, with 4.75 mm lumens on one side to hold to the hub guide hypotubes 13. A proximal guide disk 19 may have 3.0 mm lumens on the proximal side, to allow the pusher rod 9 to slide freely. The distal guide disk 19 may have 0.020″ lumens on the distal side, to help guide and center entry of the hub hypotubes 8 at the distal end of the hub guide hypotubes 13.

The catheter 1 may have a tool channel 20 for biopsy tools (1.8 mm OD) and endoscope cameras to pass through. The catheter shaft 5 may have a central lumen for tool passage with 0.101″ ID, through the entire length of the catheter. The tool channel tube 20 may have a 0.087″ ID to fit 1.8 mm biopsy tools, and a 0.098″ OD to be inserted into the proximal end of the catheter shaft 5. The tool channel tube 20 may be a braid reinforced pebax extrusion, which provides flexibility upon exit from the hub body 6, and kink resistance to resist buckling when tools are pushed through. Thus, advantages are provided that include alignment with actuator 2, alignment of the pusher rod 9 and hub hypotubes 8, alignment with actuator 2, protection of push-pull components, ease of manufacture, and tool channel functionality. The hub guide channels 32 position the proximal ends of the pusher hypotubes 9 at the same pitch diameter substantially parallel to the actuator clamps 6, for ease of loading/clamping. The hub guide channels 32 ensure that the pusher hypotubes 10 are pushed and pulled in-line with the hub hypotubes 8, thus preventing exerting a pushing force on the pusher hypotubes 10 at an extreme angle and damage thereof. The hub guide channels 32 may cover and protect the hub hypotubes 8 from external damage. Regarding manufacturability, straight, uniform diameter hub guide channels 32 may be manufactured at low cost, e.g., extruded tubing, with the single-part hub body 6 minimizing the number of parts in the hub assembly. The multi-part hub body 6 with guide hypotubes 13 and guide disks 19 adds parts but is easily manufactured and does not require a secondary operation or complicated molded parts to accommodate a tool channel 19 exit port. The tool channel tube 20 provides functionality by allowing use of endoscopes and biopsy tools, and with a luer fitting, for suction and irrigation.

The hub body 6 may include at least two guide discs 19, with a distal guide disc 19 of the at least two guide discs 19 surrounding a proximal part of a tool channel 20, with a distal end of the tool channel 20 extending through the distal section of the continuum robot, and with a proximal guide disc 19 of the at least two guide discs 19 being longitudinally aligned with the distal guide disc 19 and a plurality of hub guide hypotubes 13 traversing the at least two guide discs 19. The robotic apparatus may also include a plurality of hub guide hypotubes 13 that symmetrically surround the hub body 6 and a plurality of pusher hypotubes 10, with the plurality of hub guide hypotubes 13 extending from a distal end of the hub body 6 to respective distal ends of the plurality of pusher hypotubes 10. The respective distal ends of the plurality of pusher hypotubes 10 may longitudinally align with the plurality of hub guide hypotubes 13, and the respective distal ends of the plurality of pusher hypotubes 10 may overlap respective proximal ends of the hub guide hypotubes 13. The robotic apparatus may also include a guide disc 19 located adjacent to a hub cone 30 of the hub body 6 and, within the extension from the distal end of the hub body 6 to the respective distal ends of the plurality of pusher hypotubes 10, the plurality of hub guide hypotubes 13 may transition between two pitch diameters, with the first pitch diameter of the two pitch diameters being located at or substantially adjacent to a catheter shaft 5 contacting the distal end of the hub body 6, and the second pitch diameter of the two pitch diameters being located at or substantially adjacent to guide disc 19.

To prevent hub hypotube buckling at the pitch diameter transition, the at least one driving wire 4 may extend through one hub guide hypotube 13 of the plurality of hub guide hypotubes 13, and the hub guide hypotube 13 may be affixed to the hub body 6 and may be configured to maintain contact with the hub body 6 when the actuator 2 drives the at least one driving wire 4.

Regarding connection to the actuator clamp 7, the OD of the pusher hypotube 10 may be too small or may not provide sufficient stiffness, clamp strength, and/or durability. As described above, the maximum size of the pusher hypotube 10 is limited by available hypotube 10 size, since the pusher hypotube needs to have low friction with the spring. The actuator clamp may require a larger diameter than the maximum size of the pusher hypotube 10. Additionally, if the actuator clamps 7 are located far from the hub body, and/or the wire 4 is pushed with high force, a larger diameter pusher rod may be needed to avoid buckling/damage. The actuator clamp 7 may also require a specific material or feature on the proximal end of the pusher rod to achieve high clamp strength.

FIG. 22 illustrates an uninsulated clamp rod.

When the actuator clamp 7 is constructed of conductive material, electrical insulation may not be provided. FIG. 22 illustrates a clamp rod 23, pusher reinforcement hypotube 11 and pusher hypotube 10 that may not meet the requirements for electrical isolation for connection to the actuator clamp 7 of actuator 2. See, e.g., IEC 60601-01. To achieve desired protection, the drive wire 4 and actuator clamp 7 a minimum of 4.0 mm creepage and 2.5 mm clearance/air gap distance should be provided, with insulation that may withstand 1500 VAC. To achieve 2MOPP, clearance/creepage distances may be doubled and the insulation requirement may be 4000 VAC.

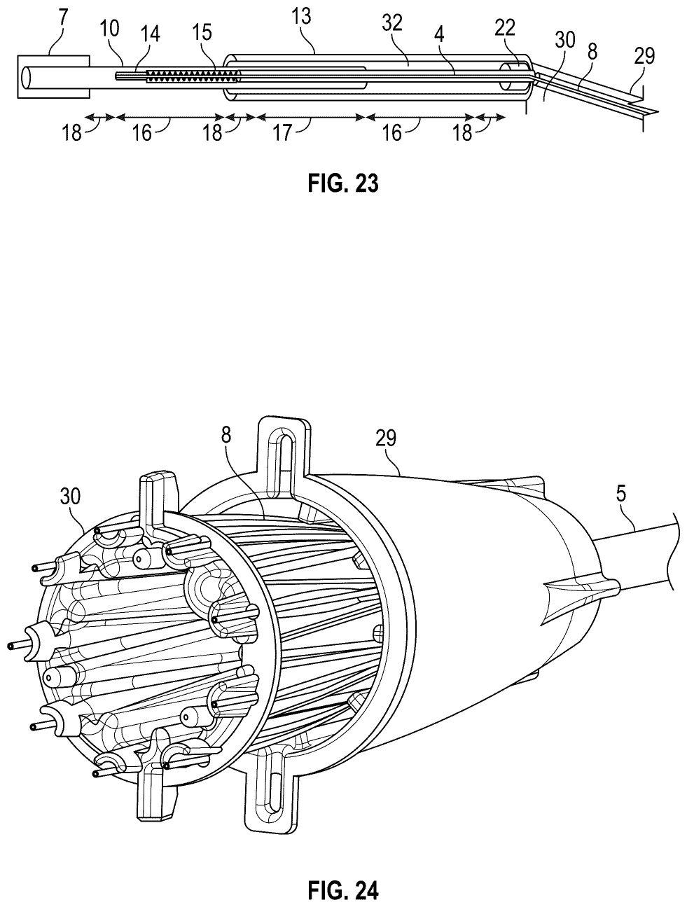

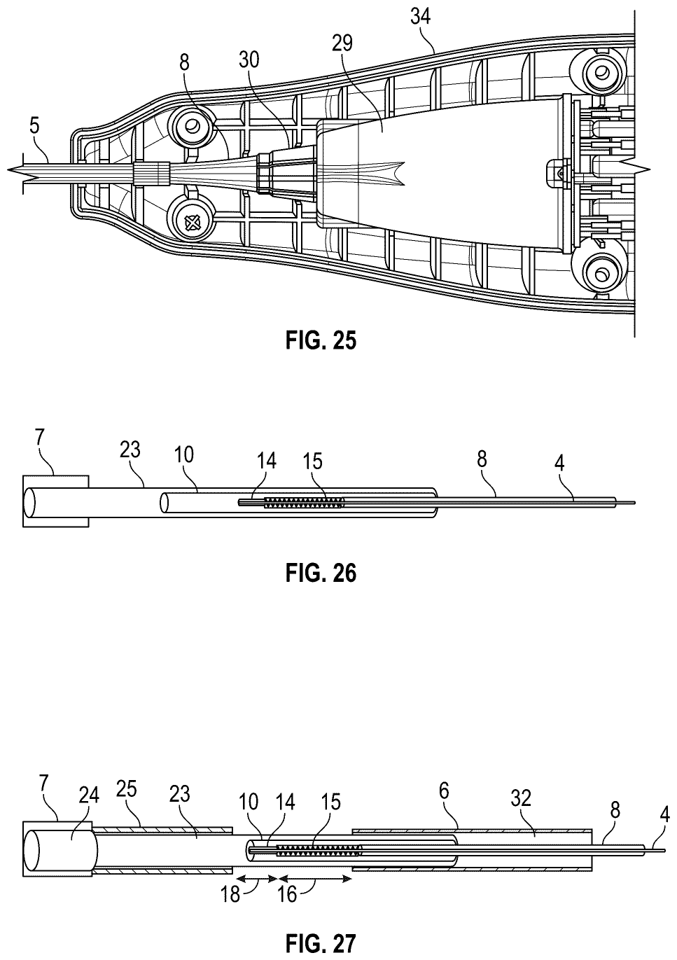

FIG. 23 is a cutaway profile view of the hub body with the hub insert tube, according to the present disclosure. FIG. 24 is a rear profile view of an elastomeric cone cover being slid over a hub cone, according to the present disclosure. FIG. 25 is a cutaway profile view of a cone cover being compressed by a catheter outer shell, according to the present disclosure.

As illustrated in FIG. 23, a hub insert tube 22 is provided at distal end of a hub guide channel 32. As illustrated in FIG. 15A, the cone cover 29 may be configured to affix, by compression, hub hypotubes 8 to the curved surface of the hub cone 30. That is, the cone cover 29 may stretch over the hub hypotubes 8 to compress them against the hub cone 30, and/or may be compressed between the catheter's outer shells 34 (FIG. 25). As illustrated in FIG. 23, as the hub hypotube 8 enters the distal end of the hub guide channel 32, the hub insert tube 22 straightens and supports each hub hypotube 8.

An insert tube provided at the distal end of hub guide channel 32 closely fits inside an ID of the hub guide channel 32, and fits over the OD of the hub hypotube 8. The insert tube may be bonded to the hub hypotube 8 and the proximal end of the insert tube 22 may be positioned with one push stroke length 16 of clearance from the distal end of the pusher hypotube 10. The length of the insert tube 22 supports and straightens the hub hypotube 8 as it exits the curved hub cone 30.

The cone cover 29 affixes the hub hypotubes 8 to the hub cone 29. The cone cover 29 may have different affixing mechanisms, depending on material. The cone cover 29 overlaps the proximal end of the hub cone 30, and attaches the hub hypotubes 8 to at least the upper curved surface of the hub cone 30. The hub cone 30 may have grooves corresponding to the OD of each hub hypotube 8, such that an outer surface of each hub hypotube 8 is flush with the outer surface of the hub cone 30. A rigid cone cover may lock the hub hypotubes 8 into grooves in the hub cone 30. The cone cover 29 fits closely to the hub cone 30 surface. The cone cover 29 may have a locking feature which prevents the cone cover 29 from moving in the direction of pushing of wires 4.

An elastomeric hub cone cover may stretch over the hub hypotubes 8 to compress them against the hub cone 30. The hub cone cover 29 may have partial grooves corresponding to the hub hypotubes 8, such that the hub hypotubes 8 are slightly raised from the hub cone 30 surface. The cone cover 30 may be an elastomeric material with an undersized fit to the hub hypotubes 8, such that the cone cover 29 may stretch over and compress the hub hypotubes 8 against an outer surface of the hub cone 30. The wall thickness, undersized fit, and elasticity of the cone cover 29 are sufficient to prevent the hub hypotubes 8 from puncturing/tearing through or over-expanding the cone cover 29 and to prevent buckling.

The hub cone 30 may have partial grooves extending longitudinally along a surface thereof for the hub hypotubes 8, such that the hub hypotubes 8 are slightly raised from the hub cone 30 surface. The cone cover 29 is a compressible material, with an ID undersized compared to the hub cover 30 and hub hypotubes 8. Also, the catheter 5 may have an outer shell sized to closely fit or undersized to the OD of the cone cover 29, such that the cone cover 29 is compressed by closing the shells. The cone cover 29 may be sufficiently thick and rigid/incompressible to prevent the hub hypotubes 8 from buckling due to pushing force on the wires 4 imposed by the actuator 2.

By way of non-limiting example, the insert tube 22 (FIG. 23) may be 0.020″ ID and 3.0 mm OD. The length of the hub insert tube 22 may be 5 mm, and the hub body length may be 47 mm, to add length for the hub insert tube 22. The cone cover 29 may be molded TPU and the hub cone 30 may be molded 72D pebax. The hub cone 30 may have grooves that align with ridges on the inside of the cone cover 29 (FIG. 26). Additionally, the cone cover 29 may have two loops for locking over the corresponding tabs on the hub cone 30. Thus, advantages are provided that include prevention of buckling of small hub hypotubes, improved manufacturability, and limited stroke distance. Regarding prevention of buckling, the insert tube 22 centers and straightens the hub hypotube 8 as it exits the curved portion of the hub cone 30, thereby preventing the hub hypotube 8 from buckling in areas that are otherwise unsupported. The insert tube 22 may create a fixed end condition rather than free/unconstrained and minimizes the length of unsupported hub hypotube 8. The cone cover 29 prevents hub hypotube 8 buckling along the curved cone surface when the drive wires 4 are pushed. Regarding manufacturability, the cone cover 29 attaches the hub hypotubes 8 to the hub cone 30 with minimal parts and no adhesives. Thus, the cone cover 29 may be easily removed/replaced due to the reversible attachment to the hub cone 30. The cone cover 29 may also be used as a fixture to hold the hub hypotubes 8 in place for the reflow process, as described herein. Regarding limiting stroke distance, the insert tube 22 creates a hard stop which limits the maximum push stroke 16 of the pusher rod 9.

FIG. 26 is a cutaway profile of a clamp rod assembly, according to the present disclosure.

The clamp rod 23 includes a hollow extending through a longitudinal length thereof, with an opening through with a proximal end the pusher hypotube 10 is inserted, for the clamp rod 23 to fully cover the pusher hypotube 10. An ID of the clamp rod 23 fits closely over an OD of the pusher hypotube 10. The clamp rod 22 may be bonded to the distal end of the pusher hypotube 10. The clamp rod proximal end 22 may be clamped by the actuator clamp 6. The proximal end of the pusher hypotube 9 may be shortened to end after the wire attachment location 14. The clamp rod 23 may be constructed of, or coated with, an electrically insulating material. A distance from the distal end of the pusher hypotube 10 to the actuator clamp 7 and a distance from the proximal end of the pusher hypotube 10 to the actuator clamp 7 may be longer than distances for creepage/clearance, as necessary for the clamp rod 23 to electrically isolate the pusher hypotube 10 from the actuator clamp.

The pusher hypotube 10 may be a 21RW hypotube (0.032″ OD/0.020″ ID), 61 mm length. The 61 mm length includes 10 mm for the wire attachment 14, 30 mm for the compression spring 15, and 21 mm for the pull stroke length 16 and minimum overlap length 17. The clamp rod 23 may be a clear polycarbonate tube, with 1.0 mm ID/2.5 mm OD, 91 mm length. The pusher hypotube 10 may be bonded to the clamp rod 23 with Loctite 4311 UV adhesive. The clamped length of the actuator clamp 7 may be 20 mm from the proximal end of the clamp rod 22. The required creepage distance for electrical isolation between the catheter drive wire 4 and the actuator clamp 7 may be 4.0 mm, and the clearance distance may be 2.5 mm. The proximal end of the clamp rod 23 may be filled with adhesive, and the distance between the distal edge of the actuator clamp 7 and the distal end of the clamp rod 23 may be 71 mm, to far exceed the creepage/clearance requirement. As can be appreciated, the dimensions provided herein are exemplary and may be modified to accommodate other pathways or desired points of interest in any given surgical circumstance.

The clamp rod 23 arrangement, as discussed herein, provides advantages that include improved stiffness/durability of the pusher rod 8, improved electrical isolation, and shorter hub length. The larger non-hypotube clamp rod 23 increases stiffness and kink resistance of the pusher rod 9, which is advantageous if the distance to the actuator 2 is far, where long thin hypotubes could easily buckle or be damaged, since the pusher hypotube 10 does not need to extend the entire distance to the clamp 7. The clamp rod 23 insulated drive wire 4 from and the nearest actuator part, i.e., clamp 7. With conventional systems, a larger diameter clamp rod 23 had to be attached to the pusher hypotube 10 at a location that is further outside the hub body 6, creating a weak/unprotected transition area and increasing the length of the assembly.

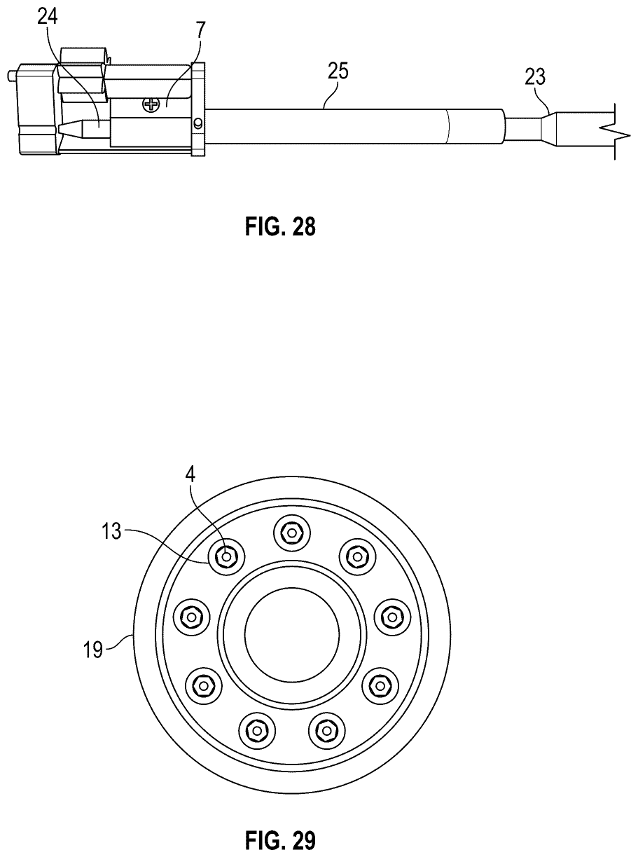

FIG. 27 is a profile view of a hypotube sleeve with actuator guide tube, according to the present disclosure. FIG. 28 illustrates an internal arrangement of the hypotube sleeve inserted into the actuator, according to the present disclosure.

FIGS. 27 and 28 illustrate a clamp rod 23 for a miniaturized actuator with sheet metal clamps, to provide electrical isolation. The clamp mechanism may have a non-uniform diameter clamp rod 23. As illustrated, hub guide hypotubes 13 closely fit over the clamp rods 23, due to the limited space. In the event that the actuator clamp 7 does not perfectly align with the guide hypotube 13, the clamp rod 23 is pushed against an inner surface of the guide hypotube 13, making catheter loading difficult, especially with multiple clamp rods to be loaded simultaneously. As illustrated, a hypotube clamp sleeve 24 is provided that covers the clamped proximal end of the clamp rod 23, providing increased electrical shielding.

Actuator 2 may include sheet metal compression clamps with guide tubes 25 which extend further over the clamp rods 23 than the section clamped by actuator clamp 7. The actuator clamp 7 may compress the clamped end of the clamp rod 23 in a metal clip, which a cam gear locks in place. The actuator guide tube 25 may fit closely over a diameter of the actuator clamp 7. The clamp sleeve 24 may be a metal hypotube. An ID of the clamp sleeve 24 may fit over the OD of the clamp rod 23, and the clamp rod OD fits into the actuator clamp 7. The clamp sleeve 24 may have sufficient wall thickness to be clamped without deformation. A distal end of the clamp sleeve 24 may be located adjacent to the distal end of the section clamped by actuator clamp 7. The clamp rod 23 has a smaller diameter than the clamp sleeve 23, and has more clearance with the actuator guide tube 24. As described herein, the clamp rods 23 may slide into a hub body 6 via the hub guide channels 32.

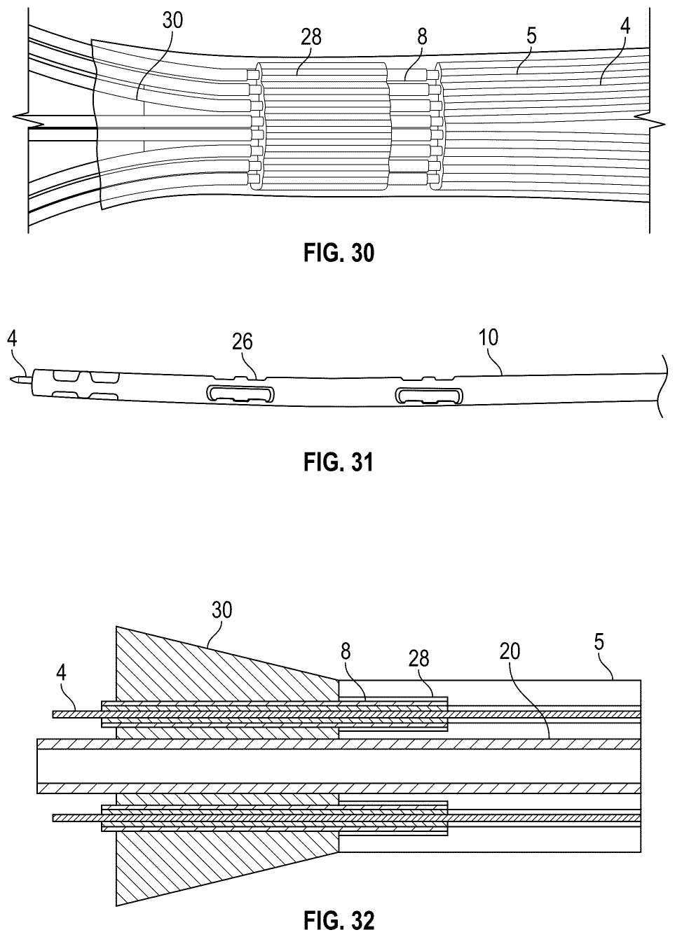

By way of non-limiting example, the actuator sheet metal clamp may have a 3.10 mm clamped diameter, and 20 mm clamped length. The actuator guide tube 24 may extend 36 mm past the clamped section over the clamp rod 23. The guide tube 25 may have a 3.25 mm ID, close to the 3.10 mm clamped diameter. The clamp rod 23 may be a polycarbonate tube with 2.3 mm OD/1.0 mm ID. The clamp sleeve 24 may be a stainless steel 11TW hypotube with a 0.120″/3.05 mm OD to fit into the actuator clamp 6, and a 0.100″/2.54 mm ID, to fit over the clamp rod 23. The clamp sleeve 24 shown in FIG. 30 is tapered to a rounded point at the proximal end, for improved alignment of the clamp rods 23 into the guide tubes 25.

The distance between the distal end of the guide tube 25 and the proximal end of the hub body 6 may be at least one push stroke length 16, so that the guide tubes 25 do not collide with the hub body 6 at the maximum push stroke. The minimum creepage distance for electrical isolation between the catheter drive wire 4 and the actuator clamp 7 is 4.0 mm, and the clearance distance is 2.5 mm. The clamp rod 23 may be filled with adhesive to block the proximal end from the uninsulated drive wire 4. The distance between the distal edge of the actuator guide tube 25 and the distal end of the clamp rod 23 may be 71 mm, which far exceeds the creepage/clearance requirement.

Various advantages include greater precision of the OD of the sheet metal clamp, reduced deformation/breakage, ease of loading, more compactness, and improved insulation. The a tight tolerance OD of the hypotube sleeve 24 reduces cost without reducing strength. Also, the narrower diameter of the clamp rod 23, as compared to the OD of clamp sleeve 24, allows the clamp rods 23 to easily slide into the guide tubes/clamp with low friction, and the tapered point of the clamp sleeve 23 also helps load multiple clamp rods 22 into narrow guide tubes 24 simultaneously. In addition, a reduced distance between the actuator and catheter is obtained without loss of functionality. In conventional systems, the clamp rod is attached to the pusher hypotube outside the hub body. To achieve the minimum creepage distance between the actuator guide tube and the exposed pusher hypotube, the actuator clamps had to be distanced farther away from the hub body. In contrast, in the present disclosure, the pusher hypotube 10 is completely covered by the clamp rod 23, reducing the distance between the actuator 2 and the catheter hub body 6.

FIG. 29 is a cutaway longitudinal view illustrating non-circular profiles of clamp rod, according to the present disclosure.

The longitudinal cutaway view of FIG. 29 illustrates non-circular profiles of clamp rod. The clamp rods 23 are non-circular and are configured to slide into hub guide channels 32. Hub guide disks 18 may have orienting features for alignment/locking of the non-circular clamp rod 23.

Regarding attachment of pusher rod to the drive wire, clamp rod 23 is provided that minimizes friction and prevents twisting of the drive wires. For a small diameter catheter with small nitinol drive wires, it is difficult to create a strong, consistent, and precisely located bond between the pusher rod 9 and drive wire 4. Attaching small wires with consistently high tensile strength is difficult, especially for nitinol wire, which is difficult to bond using typical bonding methods such as adhesives or soldering. Conventionally, adhesive bonding may be used to attach the drive wires to the support sleeves. However, the small wire diameter and small/inconsistent gap width make it difficult to achieve a consistent greater than 20 N wire attachment strength. Since the hypotubes are opaque, it was also difficult to tell how much of the wire was being adhered.

Additionally, adhesive bonding creates its own failure modes, such as adhesive wicking into hypotubes, springs, and onto drive wires, and creating inconsistent push stroke length and wire friction. As a non-limiting example, adhesive-bonding the 0.0095″ drive wires to 0.0115″ ID support sleeves provided inconsistent results. Adhesive-bonded tensile strength ranged from 15 to 50 N. In comparison, crimped wires to support sleeves had consistent bond strengths of 55 N±1.5 N. The wire attachment also needs to be accurately located, since the location of the wire attachment is the endpoint for the compression spring 15. If the bond location is inconsistent, the push stroke of the catheter may be shortened.

As described herein, the clamp rods 23 are non-circular and slide into respective hub guide channels 32. As a non-limiting example, the clamp rod 23 may be a polycarbonate tube with 1.0 mm ID/2.3 mm maximum OD, having a hexagonal cross-section. The hub guide channels 32 may be 2.5 mm ID/4.0 mm OD, and the proximal hub guide disk 19 may have a flat feature 19A (FIG. 5) to lock the orientation of the clamp rods 23, providing advantages that include reduced clamp rod friction with hub channels, with the profile of the non-circular clamp rod 23 minimizing surface contact between the clamp rod 23 and hub guide channel 32 to reduce friction. Drive wire twisting is also prevented, with the clamp rod profile 23 being oriented/aligned with a specific orientation with respect to the hub body 6 and the catheter shaft 5. Providing an orienting feature on the hub guide disks 19 prevent the non-circular clamp rods 23 and drive wires 4 from being twisted, which may damage the distal bending section 102.

FIG. 30 is a profile view of a connection between a catheter and hub with exposed drive wires. FIG. 31 is a profile view of a pusher hypotube with selective crimps according to the present disclosure.

As shown in FIG. 30, conventional connections leave gaps between the hub hypotubes 8 and the catheter shaft 5 along a longitudinal space that fails to support the drive wire 4. As described above, the present disclosure fully supports the drive wire 4. As shown in FIG. 31, three crimps 26 may be added at intervals along the longitudinal length. The crimps 26 attach the pusher rod to the drive wire 4, providing high bond strength, repeatability, and accuracy.

For a catheter with small diameter drive wires, a pitch diameter transition, and a tool channel, the connection between the hub and the catheter presents several challenges. To prevent wire buckling for small wires, the wires need to be continuously supported throughout the entire length of the catheter. It is difficult to maintain smooth and continuous wire support at a location where the drive wires transition from one body into a second body. Attachment points can introduce ledges where wires can catch. Different materials at the attachment points may create friction. Other concerns include irregular channel sizes, channel misalignment, and gaps where the wire is unsupported. Locations where the path of the wires curves also increase the risk of buckling, such as the transition from a pushing mechanism in a hub body from a large diameter tapering to a catheter having a small diameter. Assembly-wise it may also be difficult to create these smooth wire transitions in an accurate and repeatable way. For example, keeping the support channels for multiple wires perfectly aligned between two components requires both an accurate assembly method as well as accurately manufactured parts.

Challenges for the catheter-hub connection also exist. For example, the transition of the wire channel from the hub hypotubes to the catheter lumens must be smooth and fully covered to prevent wire buckling or friction. The hub cone, hub hypotubes, tool channel, and catheter shaft need to be bonded securely, with limited space for bonding or support. The hub hypotubes need to be supported at the base of the cone where they bend from a curved cone to a straight catheter shaft. The connection between the tool channel and catheter shaft needs to be hermetically sealed, to allow the tool channel to be used for suction/irrigation.

A gradual reduction in stiffness in the connection area is desired, from the larger/stiffer hub body to the smaller diameter, flexible catheter shaft, for catheter robustness, to avoid weak points where the catheter can easily break/be damaged.

As described herein, the drive wires 4 are attached to the pusher hypotube 10 via crimping, which may include multiple crimps 26, for example the three crimps 26 in pusher hypotube 10 shown in FIG. 31. The pusher hypotube 10 may be crimped at least at one location 26 along a length of the pusher hypotube 10. A most distal crimp 26 may be located flush with the proximal end of the compression spring 15. The crimped pusher hypotube 10 is bonded into a clamp rod 23.

As a non-limiting example, the pusher hypotube 10 is 21RW (0.032″ OD/0.020″ ID), and the drive wire 4 is a 0.0095″ diameter nitinol wire. The drive wire 4 may be attached to the pusher hypotube 10 with three crimps 26, with a most distal crimp 26 located 10 mm from the proximal end of the pusher hypotube 10. The length of the crimped section of the pusher hypotube 10 is 10 mm, for 3×2 mm wide crimps 26. The length of the pusher hypotube 10 may be 61 mm, allowing 10 mm for crimping the wire 14, 30 mm for the compression spring 15, and 21 mm overlap with the hub hypotube 8 (16 mm pull stroke+5 mm minimum overlap).

Thus, pusher rod 9 is more strongly attached to drive wire 4. Crimping creates a strong and consistent wire attachment strength for small nitinol drive wires 4, and securely holds the wires in both the push and pull directions. Multiple crimps provide additional safety in case one crimp fails. Crimping allows precise positioning of the wire attachment 14, and can be done accurately and repeatably (e.g., with fixturing) as well as a strong bond strength to clamp rod. The crimped end of the pusher hypotube 10 creates a textured/grooved surface, which improves the bond strength between the pusher hypotube 10 and clamp rod 23.

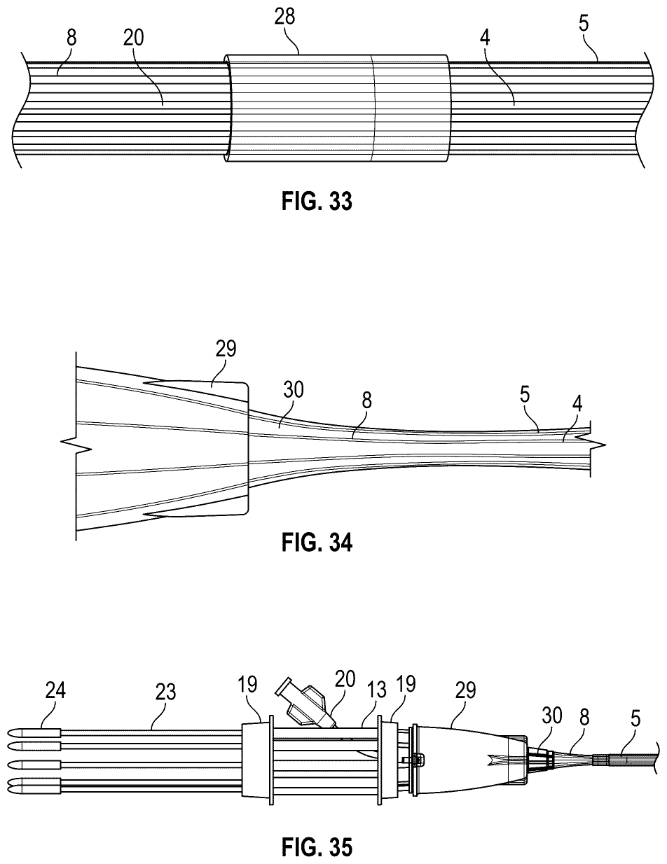

FIG. 32 is a profile view of catheter to hub connection, according to the present disclosure. FIG. 33 illustrates an assembly pre-reflow condition, according to the present disclosure. FIG. 34 shows the assembly in a reflowed condition, according to the present disclosure.

FIGS. 32 to 34 illustrate a structure for attaching hub 6 to the catheter shaft 5, including bonding together hub cone 30, hub hypotubes 8, tool channel tube 20, and catheter shaft 5.

As described herein, the catheter includes a tool channel tube 20, and has a pitch diameter transition along the hub cone 30. A proximal end of the catheter shaft 5 has lumens that are larger than the distal end of catheter shaft 5. The large lumens are sized for the OD of the hub hypotube 8. The smaller lumens are sized for the ID of the drive wire 4. The distal ends of the hub hypotubes 8 are inserted into the proximal catheter shaft lumens. The hub cone 30, tool channel 20, and catheter shaft 5 may be thermoplastic material. The hub cone 30, tool channel 20, catheter shaft 5, and hub hypotubes 8 may be thermally bonded, i.e., reflowed, together, as illustrated in FIG. 34. Mandrels may be used to keep the tool channel and wire channels open, with the tool channel mandrel being a same diameter as the ID of the tool channel tube 20, and the wire channel mandrels may be the same diameter as lumens of the catheter shaft 5 and the ID of the hub hypotube 8.

The catheter shaft 5 may have nine nitinol drive wires 4 with 0.0095″ OD, and the hub hypotubes 9 may be 304 stainless steel, 26TW hypotubes, with 0.012″ ID/0.018″ OD. The tool channel tube 20 may be a single lumen 63D pebax extrusion, 0.091″ ID/0.104″ OD. The catheter shaft 5 may be a multi-lumen 72D pebax extrusion, 0.101″ ID/0.1461″ OD, with eighteen small lumens (nine used for drive wires) and a central lumen for tool passage, proximal catheter shaft lumen guide 28 may be 0.0165″ ID, and 5 mm length, and the distal lumens of the catheter shaft 5 may be 0.0125″ ID, extending all the way through the distal bending section 102. The hub hypotubes 8 may be inserted 5 mm into the proximal catheter shaft lumen guide 28. The tool channel tube 20 may be inserted into the catheter shaft 5, ending 3 mm past the proximal edge of the distal catheter shaft 5.

In the reflow process, the hub cone 30, tool channel tube 20, hub hypotubes 8, and catheter shaft 8 are reflowed all together at ˜180 C, with FEP heat shrink. During the reflow process, the inner diameter of the tool channel tube 19 and catheter shaft 4 may be supported by a 0.091″ PTFE coated mandrel, whereas the inner diameter of the hub hypotubes 7 and catheter lumens 4 may be supported by 9×0.0113″ PTFE coated mandrels, which are removed post-reflow. Several distinct advantages are provided, including improved attachment of the hub to the catheter and improved manufacturability. Regarding hub attachment to the catheter, reflowing the hub cone 30, tool channel tube 20, hub hypotubes 8, and catheter shaft 5 creates a strong attachment between all components in a space that is limited for bonding. Regarding manufacturability, reflowing all parts together can be done in a single manufacturing step, compared to the many individual bonding steps required for adhesive bonding. Reflowing is also much faster, easier to perform, and more repeatable than adhesive bonding, and no additional parts are required, while providing a robust connection. After reflowing, the entire catheter-hub connection area is a solid structure conforming to all components, with no individual joints that could fail. Reflowing also creates a gradual transition in material stiffness from the larger, rigid hub body 5 to the smaller, flexible catheter shaft 5, which greatly improved stiffness and durability of the connection area. In addition, a smooth wire transition is provided to reduce friction/buckling risk, with reflowing allowing the catheter shaft 4 material to re-form around the hub hypotubes 8 and the 0.0113″ mandrels, which creates a smooth, uniform diameter channel for the drive wire 4 to slide through, without gaps or mismatched edges. The wire channel is also sealed from contamination, allowing use of suction/irrigation while preventing entry of dust/debris.

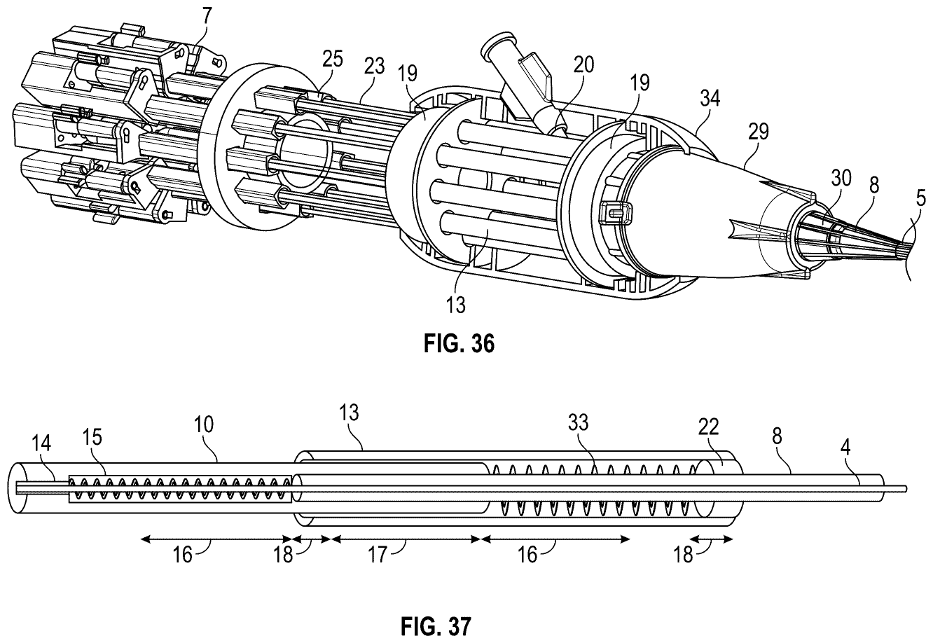

FIG. 35 is a profile view of a catheter, according to the present disclosure. FIG. 36 is a perspective view of a catheter assembly, according to the present disclosure.

As illustrated in FIG. 36, a steerable robotic catheter includes small diameter catheter shaft (4 mm) and hub hypotubes (0.018″ OD), small NiTi drive wires (0.0095″) configured to be pushed/pulled with a +/−16 mm stroke length with up to 20 N force, a wire pitch diameter increase from the catheter shaft to the actuator from 3.1 mm to 22 mm, a tool channel with an exit port through the hub body, and a miniaturized actuator with sheet metal clamps and guide tubes requiring electrical isolation, as described above.

As described above, particularly for small diameter hypotubes and long push stroke, hub hypotubes may buckle within an unsupported section at the distal end of the hub body. The steerable robotic catheter of the present disclosure supports the hub hypotubes and prevents buckling.

By way of non-limiting example, drive wires 3 may be 0.0095″ diameter nitinol wires; hub hypotubes 8 may be 304 SS 26TW, 0.012″ ID/0.018″ OD, 120 mm length; pusher hypotubes 10 may be 304 SS 21RW, 0.020″ ID/0.032″ OD, 61 mm length; and compression spring 15 may be 30 mm free length/9 mm solid length, 0.0025″ nitinol wire, 0.018″ OD, with minimum length for hypotube overlap/safety 18 of 5 mm, and maximum push/pull stroke distance 16/17 of +/−16 mm with a maximum actuator force on the wire of 20 N.

The hub body may have hub guide hypotubes 13 that are Delrin® extrusion, with 2.5 mm ID/4.0 mm OD, 52 mm length, and hub guide disks 19 that support the hub guide tubes at the actuator pitch diameter, and outer catheter shells 34 that lock the hub guide disks in place. Tool channel tube 20 may be 63D pebax extrusion, 0.104″ OD/0.091″ ID; and the tool channel tube 20 passes between the hub guide hypotubes 13 to connect to the central lumen of the catheter shaft 5. The hub cone 30 with guide channels supports hub hypotubes 8 and drive wires 4 through the transition in wire pitch diameter, from a 22 mm actuator pitch diameter 1 to 3.11 mm in the catheter shaft 5. Hub hypotubes 8 may be attached to the hub cone 30 with a cone cover 29 which locks over the hub cone 30, and hub insert tubes 22 straighten the hub hypotubes 8 at the proximal end of the hub cone 30.

Regarding an insulating clamp rod for sheet metal actuator clamp, as described above, the actuator connection may use actuator clamps 7 which are sheet metal clamps with a 20 mm clamped length, 3.10 mm clamped diameter. The actuator guide tubes 25 may extend 36 mm over the clamp rods 23 past the clamped section, with 3.25 mm ID. Electrical isolation between the catheter drive wires 3 and actuator clamps/guide tubes (7,25) may be required, with a minimum 4.0 mm creepage distance per ISO 60601-01.

Clamp rod 23 fully covers the pusher hypotube 10, and may be formed of clear polycarbonate extruded tubing, 1.0 mm ID/2.3 mm OD, 113 mm length having a hexagonal profile. Hypotube clamp sleeve 24 may cover the clamped proximal end of the clamp rod 23, and clamp sleeve 24 may be 20 mm length, 304SS 11TW, 2.54 mm ID/3.05 mm.

For a reflowed catheter-hub connection, as described above, a catheter shaft 5 may be provided of multi-lumen 72D pebax extrusion, with 3.71 mm OD/2.57 mm ID. The catheter extrusion may have larger lumens at the proximal end (0.0165″), and smaller lumens at the distal end (0.0125″). Hub hypotubes 8 may be inserted into the larger lumens in the proximal catheter shaft. Hub cone 30, catheter shaft 5, tool channel tube 20, and hub hypotubes 8 may be thermally bonded together.

FIG. 37 is a profile cutaway view of a hub support spring, according to the present disclosure.

The hub support spring 33 illustrated in FIG. 37 may be added to the arrangement of the hub insert tube 22 provided at the distal end of the hub guide channel 32, as described herein. The hub support spring 33 supports the hub hypotube 8. The hub support spring 33 may have an OD slightly smaller than the ID of the hub guide hypotube 13. The compressed length of the hub support spring 33 is at least the length of the maximum push stroke 16, with the length of the hub body 6 being increased by the compressed length of the support spring 33.

As a non-limiting example, the pusher hypotube 10 may be 21RW (0.032″ OD/0.020″ ID), the hub hypotube 8 may be 26TW (0.018″ OD/0.012″ ID), the hub guide channel 32 may have a 0.036″ inner diameter, and the hub support spring 33 may have a 0.032″ OD, with 0.005″ diameter wire. The hub support spring 33 may have a 32 mm free length and 11 mm solid length (85 coils), allowing the hub support spring 33 to be compressed 16 mm push stroke 15+5 mm for safety 17 (21 mm stroke length). The hub guide channel 32 may have 58 mm length, which includes the 21 mm pull stroke 16 plus overlap 17, 32 mm support spring 33, and 5 mm for the hub insert tube 21.

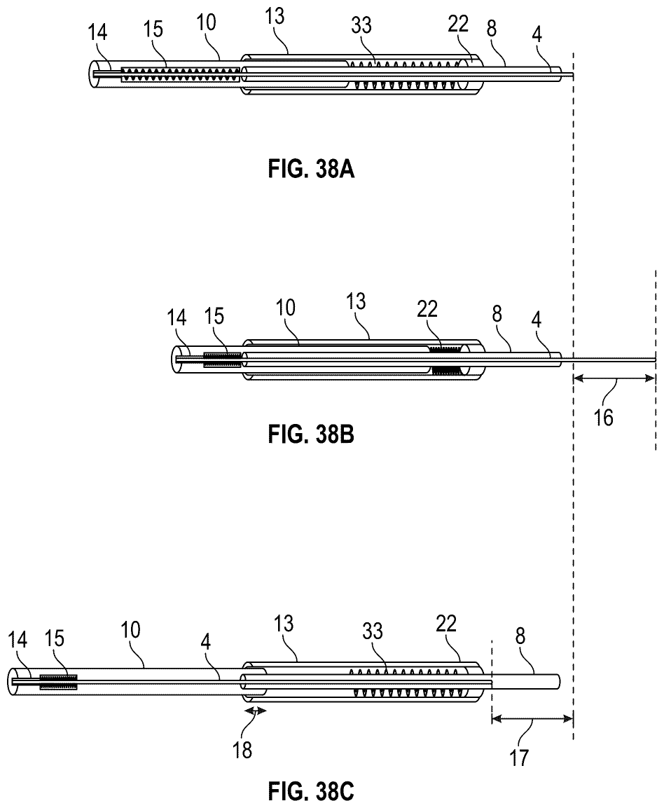

FIG. 38A to 38C illustrate a push-pull assembly with the hub support spring, according to the present disclosure.

FIG. 38A illustrates a neutral mode of the push-pull assembly. FIG. 38B illustrates the push-pull assembly in a compressed mode. FIG. 38C illustrates the push-pull assembly in an extended mode.

The push-pull assembly of FIGS. 37 to 38C provides a push-pull assembly that includes additional support for preventing buckling of hub hypotubes, without requiring additional hypotubes/assemblies and while maintaining pusher rod robustness and reduced catheter size. Advantages of addition of the hub support spring 33 include additional prevention of hub hypotube buckling. The support spring 33 fills the gap between the hub hypotube 8 and the ID of the hub guide channel 32 to prevent hub hypotube 8 buckling. Hub hypotubes 8 may buckle if pushed with excess force or are unsupported for a long push stroke 15. As such, the hub support spring 33 does not interfere with the compression spring 30 covering the drive wire 4. Compared to conventional systems, this push-pull assembly uses fewer hypotubes 8, does not have bonded assemblies, provides improved robustness of pusher rod 9, and reduces catheter size.

Thus, the present disclosure provides a push-pull assembly for operably connecting a continuum robot with a controller, with the assembly including a pusher hypotube 10 with a distal end extending towards the continuum robot, a proximal end extending towards the controller, and a hollow extending through at least a part of a longitudinal length thereof. The assembly may also include a support sleeve 8 with a proximal end slidably maintained within the hollow of the pusher hypotube 10. Also, the assembly may include a driving wire 4 with a distal end of the continuum robot, a proximal end affixed within or to the pusher hypotube 10, and a portion extending through the support sleeve 8.

The push-pull assembly may also include a hub body 6 configured to maintain a distal end of the pusher hypotube 10; a hub guide hypotube 13; and at least one guide disc 19 configured to support the hub guide hypotube 13. The controller may include at least one of an actuator and a handle configured to receive user manipulation.

The push-pull assembly may also include a clamp rod 23 configured to at least one of removably attach to the controller and/or electrically isolate the pusher hypotube 10 from the controller 2. The pusher hypotube 10 may be configured to affix to the clamp rod 23 and the clamp rod 23 may include a hollow configured to cover a proximal end of the pusher hypotube 10.

The push-pull assembly may also include a deformable member 15 positioned within at least a portion of the hollow of the pusher hypotube 10. The deformable member 15 may surround at least a portion of the driving wire 4. By surrounding the portion of the driving wire 4, the deformable member 15 provides an expanded effective diameter of the driving wire 4, thus eliminating space within with the driving wire 4 may buckle upon application of an excess pushing force, and preventing internal buckling.

In an aspect of the present disclosure, in response to application of a pushing force on the proximal end of the pusher hypotube 10, the deformable member 15 exerts a pushing force on the support sleeve 8. In response to application of a pulling force on the proximal end of the pusher hypotube 10, the deformable member 15 separates from the proximal end of the support sleeve 8 while the proximal end of the support sleeve (hub hypotube 8) is maintained within the hollow of the pusher hypotube 10.

The deformable member 15 may be configured to maintain at least one of a uniform inner diameter and/or a uniform outer diameter when compressed. The deformable member 15 may be formed of metal with a predetermined pattern that surrounds at least at part of the hub hypotube 8. The predetermined pattern may be one or more of a spring, a helix and/or a striated member.

An aspect of the present disclosure provides a robotic apparatus that may include a continuum robot 100 with a driving wire and a distal section configured to change a posture and/or a pose in response to driving of the driving wire 4 by a controller. The robotic apparatus may include a hub body 6; a pusher hypotube 10; and a support sleeve extending at least partially through or across the hub body 6. Between the controller 2 and the hub body 6, the driving wire 4 may be surrounded and supported by at least one of the support sleeve and the pusher hypotube 10.

The driving wire 4 may extend through a hub guide hypotube 13 that maintains contact with the hub body 6 when the controller 2 drives the driving wire 4. The controller may include at least one of an actuator and a handle configured to receive user manipulation. An aspect of the present disclosure may also include at least one crimp configured to secure the pusher hypotube 10 to the support sleeve.

An aspect of the present disclosure may include a deformable member 15 with the driving wire extending from the continuum robot 100 through or across the hub body 6 to the controller 2. The deformable member 15 may be provided in the pusher hypotube 10. The deformable member 15 may surround at least a portion of the driving wire 4. The support sleeve may be configured to at least one of guide and support at least a portion of the driving wire 4 from a shaft 5 of the continuum robot 100 past a distal end of the pusher hypotube 10. A proximal end of the support sleeve may extend into a distal end of the pusher hypotube 10 and contact the deformable member 15.

The pusher hypotube 10 may maintains a minimum overlap distance 18 over the support sleeve when the controller retracts the driving wire 4.

An aspect of the present disclosure may also include a clamp rod 23 having a hollow extending through at least a part of a length thereof, with a portion of the pusher hypotube 10 being affixed in the hollow of the clamp rod 23, and a hypotube clamp sleeve 24 configured to fix to the controller 2 and cover a proximal end of the clamp rod 23 that electrically isolates the pusher hypotube 10 from the controller 2.

An aspect of the present disclosure may also include a hub insert tube 22; a hub guide hypotube 13 with a hollow extending through a longitudinal length thereof; and a deformable member (hub insert spring 33). Also, the hub insert tube 22 may be configured to be positioned in the hollow of the hub guide hypotube 13, at a distal end of the one hub guide hypotube 13. In addition, the deformable member 33 may be is positioned in the hollow of the hub guide hypotube 13, with a distal end of the deformable member 33 contacting the hub insert tube 22. A proximal end of the deformable member 33 may contact a distal end of a pusher hypotube 10, and the distal end of the pusher hypotube 10 may be movable along at least a part of the hollow of the hub guide hypotube 13. In addition, a proximal end of the support sleeve may extend into the distal end of the pusher hypotube 10 and may contact a distal end of another deformable member 15.

Each of the deformable member 33 and the another deformable member 15 may be formed of metal with a predetermined pattern that surrounds at least at part of the hub hypotube 8. The predetermined pattern may be one or more of a spring, a helix and/or a striated member.

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD) TM), a flash memory device, a memory card, and the like. An I/O interface can be used to provide communication interfaces to input and output devices, which may include a keyboard, a display, a mouse, a touch screen, touchless interface (e.g., a gesture recognition device) a printing device, a light pen, an optical storage device, a scanner, a microphone, a camera, a drive, communication cable and a network (either wired or wireless).

The detector interface also provides communication interfaces to input and output devices. The detector may include, for example a photomultiplier tube (PMT), a photodiode, an avalanche photodiode detector (APD), a charge-coupled device (CCD), multi-pixel photon counters (MPPC), or other. Also, the function of detector may be realized by computer executable instructions (e.g., one or more programs) recorded on a Storage/RAM.

### REFERENCE NUMBERS

| | |
|---|---|
| Catheter | 1 |
| Actuator | 2 |
| Push/pull drive wires | 4 |
| Catheter shaft | 5 |
| Hub body | 6 |
| Actuator clamp | 7 |
| Hub hypotube | 8 |
| Pusher rod | 9 |
| Pusher hypotube | 10 |
| Pusher reinforcement hypotube | 11 |
| Outer guiding hypotube | 12 |
| Hub guide hypotubes | 13 |
| Wire attachment location | 14 |
| Resilient member | 15 |
| Push stroke distance (max) | 16 |
| Pull stroke distance (max) | 17 |
| Minimum hypotube overlap | 18 |
| Hub guide disks | 19 |
| Flat feature | 19A |
| Tool channel tube | 20 |
| Hub extrusion | 21 |
| Hub insert tube | 22 |
| Clamp rod | 23 |
| Hypotube clamp sleeve | 24 |
| Actuator guide tube | 25 |
| Wire crimp location | 26 |
| Crimp hypotube | 27 |
| Proximal catheter shaft lumen guide | 28 |
| Cone cover | 29 |
| Hub cone | 30 |
| hub guide channel | 32 |
| Support spring for hub hypotube | 33 |
| Catheter outer shell | 34 |
| Medical device system | 40 |
| Positioning cart | 44 |
| Navigation software | 46 |
| Operation console | 50 |
| Base stage | 52 |
| Continuum robot | 100 |
| Distal bending section | 102 |
| Middle bending section | 104 |
| Proximal bending section | 106 |

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A push-pull assembly for operably connecting a continuum robot with a controller, the assembly comprising:
a pusher hypotube with a distal end extending towards the continuum robot, a proximal end extending towards the controller, and a hollow extending through at least a part of a longitudinal length thereof;
a support sleeve with a proximal end slidably maintained within the hollow of the pusher hypotube; and
a driving wire with a proximal end affixed to the pusher hypotube and a portion extending through the support sleeve.

2. The push-pull assembly of claim 1, further comprising:
a hub body configured to maintain a distal end of the pusher hypotube;
a hub guide hypotube; and
at least one guide disc configured to support the hub guide hypotube.

3. The push-pull assembly of claim 1, wherein the controller includes at least one of an actuator and a handle configured to receive user manipulation.

4. The push-pull assembly of claim 1, further comprising a clamp rod configured to at least one of removably attach to the controller and/or electrically isolate the pusher hypotube from the controller, wherein:
the pusher hypotube is configured to affix to the clamp rod, and
the clamp rod includes a hollow configured to cover a proximal end of the pusher hypotube.

5. The push-pull assembly of claim 1, further comprising a deformable member positioned within at least a portion of the hollow of the pusher hypotube,
wherein the deformable member surrounds at least a portion of the driving wire.

6. The push-pull assembly of claim 5, wherein, in response to application of a pushing force on the proximal end of the pusher hypotube, the deformable member exerts a pushing force on the support sleeve.

7. The push-pull assembly of claim 5, wherein, in response to application of a pulling force on the proximal end of the pusher hypotube, the deformable member separates from the proximal end of the support sleeve while the proximal end of the support sleeve is maintained within the hollow of the pusher hypotube.

8. The push-pull assembly of claim 5, wherein the deformable member is configured to maintain at least one of a uniform inner diameter and/or a uniform outer diameter when compressed.

9. The push-pull assembly of claim 5, wherein the deformable member is formed of metal with a predetermined pattern that surrounds at least at part of the hub hypotube.

10. The push-pull assembly of claim 9, wherein the predetermined pattern is one or more of a spring, a helix and/or a striated member.

11. A robotic apparatus comprising:
a continuum robot including a driving wire and a distal section configured to change a posture and/or a pose in response to driving of the driving wire by a controller;
a hub body;
a pusher hypotube; and
a support sleeve extending at least partially through or across the hub body, wherein, between the controller and the hub body, the driving wire is surrounded and supported by at least one of the support sleeve and the pusher hypotube.

12. The robotic apparatus of claim 11, wherein the driving wire 4 extends through a hub guide hypotube configured to maintain contact with the hub body when the controller drives the driving wire.

13. The push-pull assembly of claim 11, wherein the controller includes at least one of an actuator and a handle configured to receive user manipulation.

14. The robotic apparatus of claim 11, further comprising at least one crimp configured to secure the pusher hypotube to the support sleeve.

15. The robotic apparatus of claim 11, further comprising a deformable member, wherein at least one of:
the driving wire extends from the continuum robot through or across the hub body to the controller,
the deformable member is provided in the pusher hypotube,
the deformable member surrounds at least a portion of the driving wire,
the support sleeve is configured to at least one of guide and support at least a portion of the driving wire from a shaft of the continuum robot past a distal end of the pusher hypotube, and
a proximal end of the support sleeve extends into a distal end of the pusher hypotube and contacts the deformable member.

16. The robotic apparatus of claim 11, wherein the pusher hypotube maintains a minimum overlap distance over the support sleeve when the controller retracts the driving wire.

17. The robotic apparatus of claim 11, further comprising:
a clamp rod having a hollow extending through at least a part of a length thereof, wherein a portion of the pusher hypotube is affixed in the hollow of the clamp rod; and
a hypotube clamp sleeve configured to fix to the controller and cover a proximal end of the clamp rod that electrically isolates the pusher hypotube from the controller.

18. The robotic apparatus of claim 11, further comprising:
a hub insert tube;
a hub guide hypotube with a hollow extending through a longitudinal length thereof; and
a deformable member, wherein at least one of:
the hub insert tube is configured to be positioned in the hollow of the hub guide hypotube, at a distal end of the one hub guide hypotube,
the deformable member is positioned in the hollow of the hub guide hypotube, with a distal end of the deformable member contacting the hub insert tube,
a proximal end of the deformable member contacts a distal end of a pusher hypotube,
the distal end of the pusher hypotube is movable along at least a part of the hollow of the hub guide hypotube, and/or
a proximal end of the support sleeve extends into the distal end of the pusher hypotube and contacts a distal end of another deformable member.

19. The robotic apparatus of claim 18, wherein each of the deformable member and the another deformable member is formed of metal with a predetermined pattern that surrounds at least at part of the hub hypotube.

20. The push-pull assembly of claim 19, wherein the predetermined pattern is one or more of a spring, a helix and/or a striated member.
